SEQUENCE LISTING (8 sequences)

SEQ ID NO: 1 - protein, 9 aa, Influenza virus A
GILGFVFTL

SEQ ID NO: 2 - DNA, 45 bp, Artificial
  Primer
aatactagtgctagcgccgccaccatgaaaaagcctgaactcacc

SEQ ID NO: 3 - DNA, 41 bp, Artificial
  Primer
gacctcggttagaagactcatgacttctacacagccatcgg

SEQ ID NO: 4 - DNA, 41 bp, Artificial
  Primer
ccgatggctgtgtagaagtcatgagtcttctaaccgaggtc

SEQ ID NO: 5 - DNA, 41 bp, Artificial
  Primer
aatggtaccggatcctcacttgaatcgttgcatctgcaccc

SEQ ID NO: 6 - DNA, 47 bp, Artificial
  Primer
atcggatccgccgccaccatggaggatgccaagaatattaagaaagg

SEQ ID NO: 7 - DNA, 37 bp, Artificial
  Primer
tattctagatcagtcctgctcctctgccacaaagtgc

SEQ ID NO: 8 - protein, 9 aa, Human cytomegalovirus
NLVPMVATV

(19) United States
(12) Patent Application Publication
(10) Pub. No.: US 2005/0129671 A1
(43) Pub. Date: Jun. 16, 2005

(54) Mammalian antigen-presenting T cells and bi-specific T cells

(75) Inventors: Laurence Cooper, Sierra Madre, CA (US); Michael Jensen, Pasadena, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 10/797,609

(22) Filed: Mar. 11, 2004

Related U.S. Application Data
(60) Provisional application No. 60/453,197, filed on Mar. 11, 2003.

(51) Int. Cl. A61K 48/00; C12N 5/08

(52) U.S. Cl. 424/93.21; 435/372

(57) ABSTRACT

The present invention is directed to mammalian bi-specific T cells and methods for using these bi-specific T cells. More specifically, the invention relates to viral specific T cells that express chimeric anti-tumor receptors. These bi-specific T cell clones are a source of effector cells that persist in vivo in response to stimulation with viral antigen, leading to long-term function after their transfer to patients with cancer and autoimmune diseases.

This application claims the benefit of prior copending U.S. Provisional Application Ser. No. 60/453,197, filed Mar. 11, 2003, the disclosures of which are hereby incorporated by reference in their entirety.

### STATEMENT OF GOVERNMENT SUPPORT

This application was made in part with Government support under Grant No. PO1 CA30206 and CA33572, funded by the National Cancer Institute, National Institutes of Health, Bethesda, Md. The federal government may have certain rights in this invention.

### BACKGROUND

The present invention generally relates to mammalian bi-specific T cells and methods for using these bi-specific T cells. More specifically, the invention relates to viral specific T cells, which express chimeric anti-tumor receptors. These bi-specific T cells form a source of effector cells that persist in vivo in response to stimulation with viral antigen, leading to long-term function after their transfer to patients, for example cancer patients.

One application of T cells bi-specific for a virus and a cancer antigen such as CD19 is in the treatment of B-lineage malignancies. For example, follicular lymphomas, one of the most common sub-types of non-Hodgkin's lymphoma (accounting for 20-30% of all cases) are neoplastic counterparts of normal germinal center CD19⁺ B cells. While these lymphomas are relatively indolent, they are generally considered incurable using conventional treatments. The median survival duration from diagnosis is 7 to 9 years. Patients tend to relapse after therapy, their response to salvage therapy of shorter duration after every relapse, eventually leading to death from disease-related causes. Patients with low complete response rates or high incidence of early relapse are at especially high risk. This group of patients in particular would benefit most from innovative approaches.

Non-transformed B cells and malignant B cells both express an array of cell-surface molecules that define their lineage commitment and stage of maturation. Expression of several of these cell-surface molecules, such as CD20 and CD19, are highly restricted to B cells and their malignant counterparts, but are not expressed on hematopoietic stem cells. Trials evaluating the antitumor activity of the chimeric anti-CD20 antibody IDEC-C2B8 (rituximab) in patients with relapsed follicular lymphoma have documented tumor responses in nearly half the patients treated, although the clinical effect from these treatments usually is transient. Despite the prolonged ablation of normal CD20⁺ B cells, however, patients receiving rituximab have not manifested complications attributable to B-cell lymphopenia. Although CD19 does not shed from the cell surface, it does internalize (Pulczynski, 1994). Accordingly, targeting CD19 with monoclonal antibodies conjugated with toxin molecules is currently being investigated in humans as a potential strategy to specifically deliver cytotoxic agents to the intracellular compartment of malignant B cells.

Chimeric immunoreceptors (also known as T-bodies) for targeting tumor antigens on the cell-surface, independent of MHC, typically combine the immunoglobulin-binding region (scFv) and Fc-region (ectodomain) with a T-cell activation domain (endodomain), such as CD3-ζ. This combination allows direct recognition of cell-surface antigens. Although capable of initiating T-cell anti-tumor activity upon cross-linking of the extracellular component, some chimeric immunoreceptors currently under consideration for clinical trials only deliver a primary activation signal through a chimeric CD3-ζ (domain or FcεRI receptor γ-chain, which may result in an T-cell activation signal that may not be fully competent, based on evidence from well-recognized transgenic mice models.

The genetic modification of human T cells to express tumor antigen-specific chimeric receptors is an attractive means of providing large numbers of effector cells for adoptive immunotherapy. One of the mechanisms by which tumor cells escape from immune recognition, such as down-regulation of major histocompatibility complex (MHC) molecules, are efficiently by-passed through use of this strategy. T lymphocytes engineered to express the recombinant receptor genes are capable of both specific lysis and cytokine secretion on exposure to tumor cells expressing the requisite target antigen. The development of strategies to prevent functional inactivation or loss of chimeric receptor-modified T cells in vivo would greatly enhance the therapeutic value of T cells in a number of scenarios.

T cells can penetrate and destroy solid tumors and execute a spectrum of tumorcidal effector mechanisms. To take advantage of this, a CD19-specific chimeric immunoreceptor has been developed that combines antibody recognition with T-cell effector functions. This was accomplished using an immunoreceptor composed of an antibody-derived CD19-specific scFv, as an extracellular recognition element, joined to a CD3-ζ lymphocyte-triggering molecule. This immunoreceptor can redirect the specificity of T cells in an MHC-independent manner and upon encountering CD19⁺ target cells, the genetically modified CTL can undergo specific stimulation for cytokine production and eradicate B-lineage lymphoma cells in model systems both in vitro and in vivo. See International Patent Application No. PCT/US01/42997, filed 7 Nov. 2001, designating the United States, and corresponding published International Patent Application No. WO 02/077029 for CD19⁺ re-directed T-cells for treating a CD19⁺ malignancy or for abrogating any untoward B cell function. Similarly, a CD20-specific chimeric immunoreceptor has been developed that combines antibody recognition with T-cell effector functions to create a CD20⁺ re-directed T-cells for treating a CD20⁺ malignancy or for abrogating any untoward B cell function. See U.S. Pat. No. 6,410,319.

Adoptive transfer of ex vivo-expanded T cells that use αβ T-cell receptor (αβTCR) to recognize opportunistic viral infections or tumor-associated antigens (TAA), have been demonstrated to persist in vivo and traffic to sites of disease leading to improved immune reconstitution. However, prior methods of identifying and expanding endogenous tumor-specific T cells that can function in vivo to eradicate established disease has been limited by two factors: (i) the difficulty of overcoming or regulating T-cell tolerance to “self” antigens and (ii) down-regulation of major histocompatibility complex MHC molecules on tumor escape-variants by tumor-specific T cells, since recognition of most TAAs is dependent on MHC glycoprotein presentation.

Although adoptive transfer of chimeric receptor-expressing peripheral blood-derived T lymphocytes has resulted in anti-tumor activity in mice, clinical results have so far been disappointing. The most germane issue appears to be that adoptively transferred chimeric T cells fail to expand and lose their function in vivo in the absence of any immune response directed against the chimeric T cells. Activation studies performed in transgenic mice have suggested that the function of chimeric receptor proteins depends on the activation status of the T cell. Signaling through chimeric T-cell receptors alone was shown to be insufficient to induce proliferation and effector function in primary T lymphocytes, unless they had been prestimulated through their native receptor. Even under these conditions, however, responsiveness was soon lost. This problem is exacerbated by the general lack of tumor cell costimulatory molecules essential for the induction and maintenance of a T-cell response.

The development of strategies to prevent functional inactivation of chimeric receptor-modified cells in vivo would greatly enhance their therapeutic value. One approach to improving the survival of infused T cells is to provide exogenous T-cell help mediated by CD4⁺ T-helper cells. The CD4⁺ helper function plays a crucial role in establishing or maintaining CD8⁺ CTL-mediated antiviral or antitumoral immunity (Brodie et al., 1999; Cardin et al., 1996; Matloubian et al., 1994), and long-term maintenance of engineered T cells is clearly improved if both CD8⁺ and CD4⁺ transduced T cells are infused, rather than CD8 cells alone (Mitsuyasu et al., 2000; Walker et al., 2000).

Another strategy to maintain functional activation of chimeric receptor-modified T cells involves using Epstein-Barr virus (EBV)-specific cytotoxic T lymphocytes (CTLs) (Rossig et al., 2002). EBV infection usually causes a mild self-limiting disease during primary infection and is nearly ubiquitous, infecting more than 90% of the world population. EBV initially enters the body through the oropharyngeal mucosa and then remains latently present in B lymphocytes where it persists for life (Rickinson and Kieff, 1996). These B cells may outgrow as immortal lymphoblastoid cell lines in vitro but are controlled by a strong immune response in vivo, mediated mostly through cytotoxic T cells. EBV-specific CTL lines generated from seropositive healthy donors (Rooney et al., 1995; Rooney et al., 1998) were transduced with a chimeric receptor gene which recognized a ganglioside antigen present on tumors of neural crest origin (Muto et al., 1989; Schulz et al., 1984) including neuroblastoma, small cell lung cancer, glioblastoma and melanoma. These transduced, EBV-specific T cells could be expanded and maintained long-term in the presence of EBV-infected cells. These T cells recognized EBV-infected targets through their conventional T-cell receptor and tumor targets through their chimeric receptor and effectively lysed both.

Although this strategy was effective in maintaining functional activation of the chimeric receptor-modified T cells, it is not conducive to modulating the number of chimeric receptor-modified T cells in vivo for the purposes of coordinating anti-tumor responses in patients, especially those with relapsed malignancies. The major drawback to using EBV-specific T cells is that neither the patient nor the investigator can control the amount of EBV antigen to which the viral-specific T cells are exposed. This may result in unpredictable stimulation of the genetically modified T cells leading to possible lack of function or to over-expansion causing potential toxicity or functional inactivation of the over-stimulated T cells. This is particularly important when the introduced chimeric immunoreceptor also targets normal tissue, because over-stimulated bi-specific T cells may cause unwelcome recognition of normal host tissues. In addition, there would be no easy way to eliminate the T cells or their activity when it was no longer desired. Thus, the art would benefit from additional strategies for maintaining functional activation of chimeric receptor-modified T cells and for coordinating anti-tumor response in patients with the goal of preventing or treating tumor recurrence. This is particularly important in the treatment of relapsed malignancies.

Therefore, there exists a need in the art for methods and materials useful for providing a source of effector cells that persist in vivo in response to stimulation with viral antigen and provide long-term function in vivo after transfer to cancer patient or other patients.

### SUMMARY OF THE INVENTION

Accordingly, the present invention is directed to bi-specific mammalian T cells and methods for using these bi-specific T cells. More specifically, the invention relates to viral specific T cells that express chimeric anti-tumor receptors. These bi-specific T cells are a source of persistent effector cells that respond to stimulation with viral antigen, allowing the cells to maintain in vivo function long-term.

In one aspect, the invention provides genetically engineered bi-specific T cells which express and bear on the cell surface membrane (a) an endogenous viral antigen receptor and (b) an introduced cancer antigen-specific chimeric T cell receptor. The chimeric immunoreceptor is a hybrid molecule composed of an intracellular signaling domain, a transmembrane domain (TM) and a cancer antigen-specific extracellular domain. In one embodiment, the T cells also co-express a fusion protein of a viral antigen and/or a drug resistance protein.

In a second aspect, the invention provides a method of treating a cancer in a mammal, which comprises administering bi-specific T cells to the mammal in a therapeutically effective amount. In one embodiment, CD8⁺ bi-specific T cells are administered to a mammal with or without CD4⁺ bi-specific T cells. In a second embodiment, CD4⁺ bi-specific T cells are administered to a mammal with or without CD8⁺ bi-specific T cells.

In a third aspect, the invention provides a method of improving the in vivo survival of bi-specific T cells through the exogenous administration of interleukin-2 (IL-2).

In a fourth aspect, the invention provides a method of abrogating any untoward or undesired B cell function in a mammal which comprises administering to the mammal CD19- or CD20-specific bi-specific T cells in a therapeutically effective amount. These untoward B cell functions can include B-cell mediated autoimmune disease (e.g., lupus or rheumatoid arthritis) as well as any unwanted specific immune response to a given antigen.

In a fifth aspect, the invention provides a method of effecting and improving persistence in vivo of bi-specific T cells in a mammal by administering to the mammal a stimulating amount of viral antigen or T-cells expressing a viral antigen recognized by the T cell receptor on the bi-specific T cell.

In a sixth aspect, the invention provides a method of effectively eliminating bi-specific T cells in vivo by withdrawing administration of the viral antigen recognized by the bi-specific T cell or with-holding viral antigen recognized by the bi-specific T cell.

In a seventh aspect, the invention provides a method of effectively eliminating bi-specific T cells. In one embodiment, the T cells express a fusion protein of a viral antigen and a drug resistance protein. For example the bi-specific T cells co-express the hygromycin/thymidine kinase fusion protein and can be eliminated in vivo by administration of ganciclovir.

In an eighth aspect, the invention provides a method of using T cells as antigen presenting cells, so as to function as a type of vaccine to deliver antigen to mammals in vivo as well as function in vitro as stimulator cells to expand antigen-specific T cells.

### BRIEF DESCRIPTION OF THE FIGURES

FIGS. 1A-1B show the bi-specificity of MP1-tetramer⁺CD19R⁺T cells.

FIGS. 2A-2B show the expression of CMV pp65mII in hygromycin-resistant U293T cells genetically modified with the DNA pEK expression vector coding for hypp65 cDNA.

FIG. 3 shows lysis of hygromycin-resistant HLA-A2⁺ U293T cells expressing hypp65 by HLA-A²⁺ CD8⁺ pp65-tetramer⁺ T-cell clone that was freshly thawed.

FIG. 4 is a schematic diagram showing DNA plasmids expressing HyMP1 and Hy. A DNA plasmid derived from pKEN was used to express the hygromycin phosphotransferase gene fused in frame to the matrix protein 1 from influenza A, designated HyMP1, under control of human elongation factor 1α promoter.

FIG. 5 is a schematic drawing of a plasmid expressing ffLucZeo.

FIG. 6 shows a chemiluminescent western immunoblot of recombinant HyMP1.

FIG. 7 provides flow cytometry histograms showing the phenotype of AP-T cells.

FIG. 8 is a series of histograms showing by flow cytometry expression of HLA-A0201⁺ tetramer loaded with GILGFVFTL (MP1 amino acids 58-66; SEQ ID NO:1) binding to CD8⁺ T cells obtained from an HLA A2⁺ donor and incubated for 21 days with and without autologous irradiated hygromycin-resistant stimulator genetically modified T cells. FIG. 8A: no genetically modified stimulator T cells were added. FIG. 8B: stimulation every 7 days with T cells genetically modified with a control plasmid expressing hygromycin. FIG. 8C: stimulation every 7 days with T cells genetically modified with a plasmid expressing HyMP1.

FIG. 9 shows fold expansion of HLA-A2⁺ T cells were co-cultured under identical conditions without AP-T cells or with AP-T cells expressing hygromycin but not MP1.

FIG. 10 shows cytokine (IFN-γ, FIG. 10A; TNF-α, FIG. 10B) production by T cells under the indicated co-culture conditions.

FIG. 11 provides histograms showing binding of specific mAbs (bold lines), relative to isotype control or unstained cells (dotted lines). The relative percentage of cells in each gate is indicated.

FIG. 12 provides a bright field image (FIG. 12A) of a T cell and a tumor cells that were docked together, and an image for analysis of capping of endogenous αβTCR (FIG. 12B) and detection of Vβ17 (FIG. 12C) using a specific biotinylated mAb. FIG. 12D shows identification of tumor cells by binding of PE-conjugated anti-CD49c, a monoclonal antibody that recognizes an α3 integrin on U251T cells.

FIG. 13 shows specific lysis of ⁵¹Cr-labeled targets CD19⁺ Daudi (FIG. 13A) or MP1⁺ HLA A2⁺ AP-T (FIG. 13B) target cells.

FIG. 14 provides data confirming that the effector T cells can recognize primary B-lineage ALL cells using lysis of ⁵¹Cr-labeled blasts incubated with MP1- and CD19-bi-specific T cells.

FIG. 15 shows specific lysis of the indicated cells by HLA A2⁺ MP1- and CD19-bi-specific T cells.

FIG. 16 provides data with respect to cytokine production by HLA A2⁺ MP1- and CD19-bi-specific T cells after incubation at 37° C. with γ-irradiated CD19⁻ K562 cells, or autologous Hy⁺ AP-T cells, HyMP1⁺ AP-T cells, CD19⁺ Daudi cells, or 1:1 mixture of MP1⁺ AP-T cells and CD19⁺ Daudi cells.

FIG. 17 shows T cell proliferation upon exposure to MP1 and/or CD19 antigens as determined by ³H-TdR incorporation.

FIG. 18 shows relative in vitro ffLuc activity from transfected and non-transfected cells as indicated.

FIG. 19 provides serial non-invasive biophotonic measurements of NOD/scid mice which received intraperitoneal adoptive transfer of γ-irradiated (FIG. 19, solid line) and non-irradiated (FIG. 19, dashed line) T cells genetically modified with the plasmid ffLucZeo-pcDNA.

FIG. 20 provides pseudocolor images representing light intensity from γ-irradiated ffLuc⁺ T cells in the peritoneum of NOD/scid mice imaged in ventral position.

FIG. 21 shows non-invasive biophotonic imaging measurements which revealed the kinetics of tumor growth before and after adoptive immunotherapy. Data are presented as photon flux for a ROI drawn over the whole mouse. Accompanying scatter graphs of tumor flux versus time and pseudocolor images of selected mice (red lines) representing light intensity from ffLuc⁺ Daudi cells in the peritoneum of NOD/scid mice serially imaged in ventral position.

FIG. 22 shows background flux measurements for the same treatment groups shown in FIG. 21. Data from mice that achieved complete remission are shown in FIG. 22B. Data from progression-free or tumor-free mice are shown in FIG. 22C.

### DETAILED DESCRIPTION OF THE PREFERRED EMBODIMENTS

The present invention is directed to bi-specific T cells and methods for using these bi-specific T cells in mammals. More specifically, the invention relates to viral specific T cells that also co-express a chimeric anti-tumor receptor. These bi-specific T cells or T cell clones are a source of effector cells that can persist in vivo in response to stimulation with viral antigen, leading to long-term function after their transfer in vivo.

Since clinical efficacy of adoptively transferred T cells depends on full activation of the T cells in vivo, it would be desirable to achieve this activation through the endogenous αβTCR. This could improve the anti-tumor activity of T cells bearing a tumor-specific chimeric immunoreceptor. T cells may be capable of antigen presentation to autologous T cells, a property which was used to generate a source of vaccine that could be used in vitro and in vivo to activate T cells through the αβTCR. Since primary human T cells bearing a CD19-specific chimeric immunoreceptor can target B-lineage malignancy, the anti-tumor potency of such genetically modified T cells can be improved in vitro and in vivo by activation though the endogenous αβTCR using autologous T cells functioning as antigen presenting cells (APCs).

The approach of this invention to solve the problem of lack of maintained activity in vivo is to generate viral-specific effector T cells that express chimeric anti-tumor receptors. These cells can persist in vivo in response to stimulation with antigen, leading to long-term function after their transfer to patients, for example patients with B-lineage lymphoma or leukemia. Therefore, an embodiment of this invention includes production of T cells with two defined specificities, for example T cells that both recognize a viral antigen such as the influenza A matrix protein 1 via the endogenous αβ T cell receptor and which are rendered specific for B-lineage lymphoma by introducing a CD19-specific chimeric immunoreceptor using molecular biological techniques.

Introduction of a CD19-specific chimeric immunoreceptor, designated CD19R, renders genetically modified human T cells specific for B-lineage leukemia and lymphoma. (Cooper, 2003). To improve the potency of adoptive immunotherapy for this disease, the invention, in one embodiment, provides a novel T-cell vaccine which uses autologous T cells expressing influenza A matrix protein 1 (MP1) as antigen-presenting (AP) cells to activate in vitro and in vivo effector T cells, and which bear a tumor-specific chimeric immunoreceptor, that interacts via the endogenous αβ T-cell receptor. In tissue culture, the MP1⁺ AP-T cells stimulate a CD8⁺ T-cell recall-response, which can be shown by class I tetramer-binding and functional assays to be specific for MP1.

The CD19-specific T cells described here proliferate in direct response to CD19 antigen. This is in contrast to the apparent lack of proliferation demonstrated by genetically modified T cells expressing chimeric immunoreceptors that also use the CD3-ζ activation domains. These cells are specific for other antigens, such as G_{D2}, a ganglioside antigen present on tumors of neural crest origin, and CD33. However, human T cells bearing a CD19-specific ζ-chain-based chimeric immunoreceptor derived from mAb clone SJ25C1 can proliferate in response to CD19⁺ stimulator cells, if CD80 is co-expressed. These differences in proliferative ability of genetically modified T cells could be explained by relative differences in affinity for antigen and/or expression levels of introduced chimeric receptor. Therefore, a lack of proliferative capacity may be overcome by stimulation through endogenous αβTCR or co-stimulation through endogenous TCR or a T-cell co-stimulatory molecule such as CD80.

After non-viral gene transfer with a DNA plasmid that expresses CD19R, co-capping, chromium release, cytokine release, and proliferation assays demonstrated that MP1-specific T cells retained specificity for MP1 and acquired specificity for CD19. These bi-specific T cells were furthermore capable of receiving additional activation signals when exposed to both MP1 and CD19 antigens. The improved T-cell activation from these sources can augment the cells' anti-tumor effect; infusion of autologous MP1⁺ AP-T cells improved the ability of adoptively transferred MP1- and CD19-specific T cells in vivo to treat established tumor in a well-accepted model.

In another embodiment, this invention provides human T cells designed as a source of vaccine to present a recombinant protein in vitro and in vivo, enabling vaccination without having to use live virus to present viral antigen. Enforced expression of desirable co-stimulatory molecules, such as MICA, may further improve the antigen presenting capacity of T cells in these methods. Since T cells can be readily expanded and genetically manipulated by methods operating in compliance with current good manufacturing practice, autologous T cells advantageously may be used as both effector cells and APCs in clinical applications for stimulating adoptively transferred bi-specific T cells in the presence of an endogenous viral-specific memory response.

The clinical value of T cells expressing chimeric immunoreceptors is improved when CD19-specific genetically modified T cells are made to expand in vivo, overcoming a defect in previous T cells for adoptive therapy that is presumably due to the inherent limitations of signaling exclusively through the chimeric immunoreceptor. Docking of the TCR with cognate antigen commences a wave of protein tyrosine kinase activation of downstream signaling pathways, which ultimately leads to the expression of genes that control cellular proliferation of mature extrathymic T cells. Thus, T-cell activation through the endogenous TCR complex drives an in vivo anti-tumor response through, for example, the CD19-specific chimeric immunoreceptor.

Without wishing to be bound by theory, the mechanism for the improved in vivo anti-tumor potency of the bi-specific T cells of the invention likely depends on multiple factors. The data here suggest that upon contact with both CD19 and MP1 antigens, MP1-tetramer⁺Fc⁺ T cells achieve a higher state of activation (demonstrated by increased proliferation and cytokine production) relative to these same effector cells interacting with either antigen alone. Further, the T cells also exhibit a reduction in antigen-dependent apoptosis. Since both sequential and simultaneous contact with the cancer (CD19) and viral (MP1) antigens results in supra-physiologic activation of these MP1-tetramer⁺Fc⁺ T cells, it is unlikely that increased adherence of a bi-specific T cell for stimulator cells expressing both antigens can fully account for the augmented cytokine production and cell proliferation. Therefore, an introduced chimeric immunoreceptor can be used to provide co-stimulation to augment the activation of T cells expressing an endogenous αβTCR with marginal affinity for a TAA.

Patients can be treated by infusing therapeutically effective doses of CD8⁺ bi-specific, cancer antigen redirected T cells in the range of about 10⁶ to 10¹² or more cells per square meter of body surface (cells/m²). The infusion can be repeated as often and as many times as the patient can tolerate until the desired response is achieved. The appropriate infusion dose and schedule will vary from patient to patient, but can be determined by the treating physician for a particular patient according to methods commonly used in oncology and the results of T cell assays which may be performed on samples of the patient's blood for monitoring purposes. Typically, initial doses of approximately 10⁹ cells/m² are useful, escalating to 10¹⁰ or more cells/m² if the patient tolerates the higher amount. IL-2 can be coadministered to expand infused cells post-infusion, if desired, in amounts of about 10³ to 10⁶ units per kilogram body weight. Alternatively or additionally, an scFvFc:ζ-expressing CD4⁺ T_{H1} clone can be co-transferred to optimize the survival and in vivo expansion of transferred scFvFc:ζ-expressing CD8⁺ T cells.

The dosing schedule may be based on known methods and information. See Rosenberg et al., 1988; Rosenberg et al., 1993a; Rosenberg et al., 1993b, the disclosures of which are hereby incorporated by reference. Any alternative continuous infusion strategy known in the art may be employed. CD19-specific redirected T cells also can be administered as a strategy to support CD8⁺ cells as well as to initiate or augment a delayed type hypersensitivity response against CD19⁺ target cells.

T cells expressing a chimeric immunoreceptor can be activated through endogenous and introduced immunoreceptors. For example, Epstein-Barr virus (EBV)-specific T cells (or T cells specific for other viruses) can be rendered specific for G_{D2} or CD19 (or any other antigen) by introduction of a chimeric immunoreceptor via retroviral transduction. Applying autologous AP-T cells to trigger bi-specific T cells has distinct advantages over using EBV antigen or alloantigen as has been attempted previously in various methods. For example, since CD19-specific T cells are unable to distinguish between normal and malignant B cells bearing CD19 antigen, controlling activation of resident genetically modified T cells by selected delivery of an exogenously applied recombinant viral antigen such as MP1 antigen rather than activating T cells using latent EBV reduces the possibility of unwanted activation of bi-specific T cells and subsequent deletion of normal cells recognized by the chimeric immunoreceptor. Furthermore, the repeated administration of allogeneic cells, which may be necessary to sustain an in vivo anti-tumor response in a clinical setting would likely lead to transfusion reactions secondary to HLA alloimmunization.

Cytotoxic T-lymphocytes (CTL) specific for influenza A nuclear matrix protein 1 (MP1) can be expanded in vitro using autologous T cell antigen presenting cells that have been genetically modified to express MP1. Expression of CD19R can render MP1-specific T cells specific for CD19 so that they not only recognize either or both MP1 or CD19 antigens, but also demonstrate supra-physiologic activation in vitro when engaging both antigens. This combination of properties can be used to improve the T cells' anti-tumor activity in vivo.

Influenza A viruses have a single-stranded, segmented negative sense RNA genome characterized by its high degree of variability and the ability to cause acute respiratory infections of humans and animals, often resulting in significant morbidity and mortality (Lamb and Krug, 1996). A large body of experimental evidence suggests an essential role for neutralizing antibodies and CD8⁺ CTLs in eliminating influenza virus and promoting recovery from infection (Askonas et al., 1982; Doherty et al., 1997; Gerhard et al., 1997; McMichael, 1994; Mackenzie et al., 1989; Zweerink et al., 1977). In mice, the CTL response to this virus is directed to a limited number of immunodominant epitopes (Bennick and Yewdell, 1988).

Similar examples of immunodominance have been described for humans. In one embodiment of the invention, viral antigen recognized by the bi-specific T cell is derived from influenza. For example, in HLA-A2⁺ donors the CTL response against influenza virus is predominantly directed to the HLA-A2-restricted epitope of the matrix protein (GILGFVFTL; MP1₅₈₋₆₆; SEQ ID NO:1) (Bednarek et al., 1991; Gianfrani et al., 2000; Gotch et al., 1987; Morrison et al., 1992). Therefore, a novel recombinant fusion protein that combines a drug-resistance gene with the MP1 gene has been fashioned was designed to function as an alternative to using live virus when generating influenza-specific T cells.

The well-characterized protein MP1 from influenza A is a convenient target antigen since from a young age almost all individuals have immunity to influenza and therefore have responsive circulating memory T cells. Furthermore, because the cellular immune responses to MP1 in HLA-A2 individuals usually responds to an immunodominant epitope (amino acid 58-66), tetramer technology can readily identify MP1-specific T cells making isolation and identification easier, for example using fluorescence activated cell sorting.

Other examples of viral antigens for which there are well-defined T cell responses include cytomegalovirus (CMV) pp65 and IE. Creating CD19-specific T cells specific for these CMV antigens are a preferred embodiment of the invention for adoptive immunotherapy after allogeneic hematopoietic stem-cell transplant (HSCT) for B-lineage malignancies because recipients of such transplants are vulnerable to tumor relapse as well as opportunistic infections due to CMV. Although viral specific T cells can be generated for any virus, one attractive feature of generating T cells specific for influenza (rather than CMV or EBV) is that the patient can receive well-timed infusions of T cells presenting influenza to modulate the number of bi-specific T cells in an effort to co-ordinate anti-tumor responses in patients with relapsed B-lineage malignancies.

The generation of viral-specific T cells has required the development of tissue culture techniques that can preferentially stimulate the expansion of desired T cells from a pool of T cells with heterogenous specificities. Endogenous influenza MP1-specific specific T cells can be expanded from influenza sero-positive volunteers using repetitive 7-day stimulation cycles with irradiated hygromycin-resistant autologous T cells genetically modified to express the fusion protein hygromycin::MP1 (HyMP1). This fusion gene codes for both the bacterial protein hygromycin phosphotransferase, permitting in vitro selection of genetically modified cells by resistance to hygromycin, and simultaneous expression of the influenza matrix protein 1 (MP1).

A flexible culturing system allows for the expansion and identification of T cells with other desired specificities. For example, autologous T cells can be genetically modified to express a fusion protein of hygromycin and pp65 in order to generate hygromycin-resistant T cells capable of expressing pp65. These T cells can then be used to expand autologous pp65-specific T cells. Hygromycin-resistant T cells genetically modified to express the gene HyMP1 are capable of presenting the MP1 protein through the class I and II pathways to CD8⁺ and CD4⁺ T cells, respectively. Furthermore, a soluble fusion protein of CMV pp65 and IE can be processed by monocytes and used to expand CMV-specific T cells from PBMC.

To safeguard patient safety, non-immunogenic selection and suicide systems, such as dimerizable Fas, may be incorporated into the system. Also, to avoid initiating a hygromycin-specific immune response from AP-T cells expressing hygromycin phosphotransferase that would delete effector cells expressing HyTK gene, a fusion gene combining neomycin and MP1 may be used. Additional components of the invention may include removal of immunogenic transgenes from the effector cells to reduce the possibility of immune-mediated elimination of the transferred T cells and inhibiting the expression of classical HLA molecules on bi-specific effector T cells to prevent antigen recognition by T cells in a recipient of adoptive immunotherapy. Antigen presentation capacity of T cells also may be improved by co-expressing additional T-cell co-stimulatory molecules such as found on professional antigen presenting cells. Generation of fusion genes does not rely on partnering the viral antigen with hygromycin. Other antibiotic-resistance genes can be used, such as neomycin phosphotransferase.

MP1-specific T cells can be generated, for example, by obtaining PBMC from an influenza sero-positive normal volunteer donor that contains ˜1% MP1-tetramer⁺ CD8⁺ circulating T cells. Endogenous influenza MP1-specific specific T cells can be expanded from these cells using repetitive 7-day stimulation cycles with irradiated hygromycin-resistant autologous T cells genetically modified to express the fusion protein hygromycin::MP1 (HyMP1). These PBMC may be incubated with irradiated MP1-presenting T cells (PBMC:T cells^{HyMP1+}) at a ratio of about 1:1 to 10:1 in the presence of low-dose (about 5 U/ml) IL-2.

Following weekly stimulations with stimulating T cells, a large population of MP1-tetramer⁺ population of MP1-specific (tetramer⁺) T cells emerges in the culture and can be isolated easily using methods known in the art. For example, PBMC from an HLA-A2⁺ volunteer donor initially containing ˜1% MP1-tetramer⁺ CD8⁺ circulating T cells, were incubated at a 5:1 ratio (PBMC:T cells^{HyMP1+}) in the presence of 5 U/mL IL-2. After 21 days of repetitive in vitro stimulations the percentage of MP1-tetramer⁺ CD8⁺ T cells increased to ˜50%, demonstrating that the HyM1 fusion protein is processed through the MHC class I pathway and the immunoreactive GILGFVFTL peptide (SEQ ID NO:1) can be presented by autologous T cells. In addition to CD8⁺ MP1-tetramer⁺ T cells, the culture conditions also expanded CD8⁺ MP1-tetramer⁻ T cells and CD4⁺ T cells. A ready supply (>10⁹) of HyMP1⁺ stimulator T cells can be maintained using repetitive OKT3-driven expansion cycles growing in the presence of cytocidal concentrations of hygromycin (0.2 mg/mL). The stimulator T cells grown in this fashion have been characterized as CD8⁺CD80⁺HLA-ABC⁺HLA-DR⁺MP1-tetramer⁻ as assessed by flow cytometry.

Alternatively, the PBMC may be repetitively incubated with soluble MP1 protein. The soluble protein is taken up and processed by the MHC machinery of monocytes presenting the antigen and resulting in stimulation and preferential expansion of MP1-specific T cells. These MP1-specific cells then can be isolated using conventional methods such as magnetic bead separation based on production of γ-IFN and their specificity for MP1 again verified.

Non-human primate and human T cells that have been genetically modified to express immunogenic proteins according to this invention are capable of antigen delivery in vivo after intravenous administration, as demonstrated in the examples appended below. These data demonstrate that autologous T cells act as APCs to stimulate a recall response in vitro against the viral antigen MP1, and that the expanded MP1-specific T cells can be rendered specific for CD19. In addition, both the endogenous MP1-specific and introduced CD19-specific immunoreceptors can activate genetically modified T cells independently. The sequential and/or simultaneous engagement of both immunoreceptors results in augmented activation of the effector cells which translates into improved potency by combining autologous MP1⁺ AP-T cells with MP1-tetramer⁺Fc⁺ T cells for treating established CD19⁺ tumors in vivo. In the absence of a physiologic CD4⁺ helper-response, the in vivo persistence of adoptively transferred CTL may be maintained with exogenous IL-2.

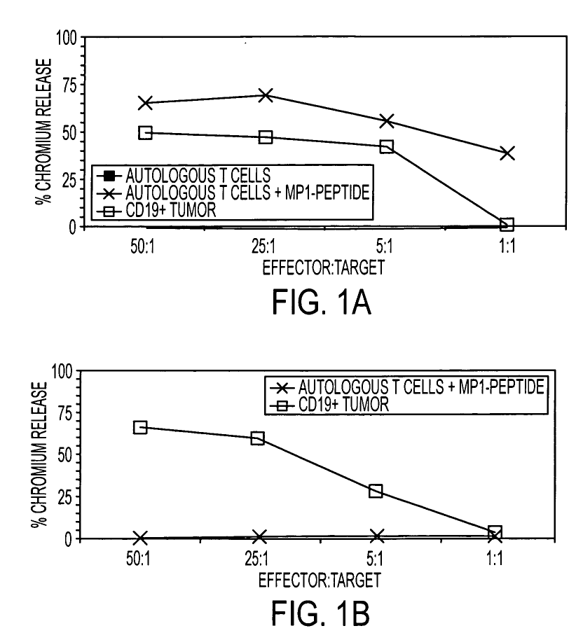

To design an in vitro system to generate antigen-presenting cells that can be used for immunization, T cells were genetically modified to express a chimeric protein of hygromycin (Hy) phosphotransferase fused to the influenza A matrix protein 1 (MP1). The fusion protein confers resistance to hygromycin, permitting in vitro selection of genetically modified cells, while the MP1-portion is processed through the T-cell proteosome apparatus. Using PBMC from an HLA-A2⁺ donor, CD8⁺MP1-tetramer⁺ T cells could be rapidly expanded by co-culture with irradiated autologous MP1⁺Hy⁺ AP-T cells. Specificity of the expanded T cells for MP1 was demonstrated by secretion of γ-IFN upon co-culture with HLA-restricted cells expressing MP1. The influenza-specific T cells then were rendered bi-specific by introduction of a chimeric immunoreceptor specific for the CD19 determinant, termed CD19R. This chimeric immunoreceptor molecule can dock with the CD19 determinant through an extracellular domain, derived from the scFv of a CD19-specific mouse mAb, leading to T-cell activation through the attached CD3-ζ chain (Cooper et al., 2002). Bi-specificity was demonstrated by chromium release assay in which the MP1-tetramer⁺CD19R⁺ T cells lysed both MP1⁺ and CD19R⁺ targets, conversely monospecific MP1-tetramer⁺ T cells and CD19R⁺ T cells killed only MP1⁺ or CD19⁺ targets, respectively. See FIG. 1. Bi-specific MP1-tetramer⁺CD19⁺CD8⁺ T cells could lyse autologous targets expressing MP1 as well as targets expressing CD19 determinant (FIG. 1A), whereas CD19⁺CD8⁺ T cells could only lyse CD19⁺ targets (FIG. 1B). The specificity for cognate antigen was demonstrated by the fact that neither effector T cell could lyse autologous T cells.

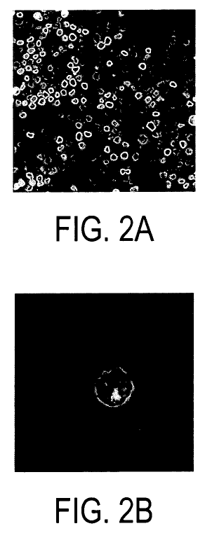

The technique of using hygromycin fusion proteins to present MP1 can be applied to other viral antigens as well. For example, fusion molecules may be constructed using a modified CMV pp65 gene combined with hygromycin phosphotransferase, designated as Hypp65. pp65 cDNA may be modified to decrease the innate protein kinase activity that is toxic to cells expressing this protein (Yao et al., 2001). See FIG. 2, which demonstrates that pp65 can be expressed in human cells grown under cytocidal concentrations of hygromycin. Cells growing in 1.6 mg/ml hygromycin B were plated onto glass slides, fixed, permeabolized and stained with mouse anti-CMV mAb using reagents and protocols from Biotest Diagnostics Corporation. Bound mAb was detected using FITC-conjugated goat anti-mouse antibody. FIG. 2A: 20×; FIG. 2B: 60×. Cells expressing pp65mII are green. Cells are counter-stained with Evans' Blue (red; FIGS. 2A and 2B) and DAPI (blue; FIG. 2A).

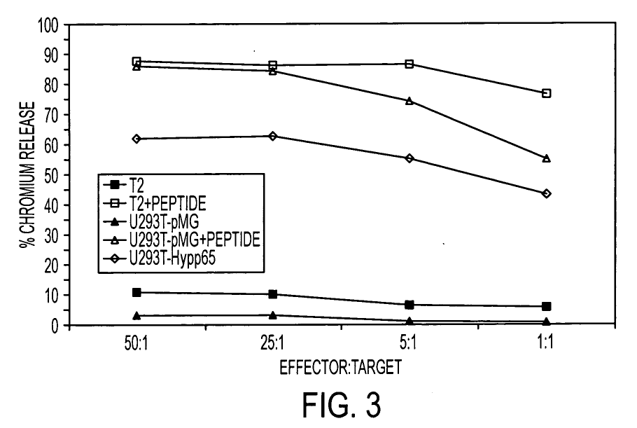

Immunoreactive pp65 proteins are presented through the MHC class I pathway since pp65-tetramer⁺ CD8⁺ T-cell clones from a HLA A2⁺ CMV sero-positive donor are able to lyse HLA A2⁺ cells genetically modified with a plasmid expressing Hypp65. See FIG. 3. Controls include hygromycin-resistant U293T cells electroporated with the pMG plasmid incubated with and without the CMV pp65 peptide NLVPMVATV (SEQ ID NO:2). T2 cells are HLA A2⁺ T-B lymphoblast hybrids incubated with and without the CMV pp65 peptide.

In one aspect, the present invention provides genetically engineered T cells which express and bear on the cell surface membrane an endogenous viral antigen receptor and an introduced cancer antigen-specific chimeric T cell receptor (referred to herein as bi-specific T cells). This chimeric T cell receptor has an intracellular signaling domain, a transmembrane domain and a cancer antigen-specific extracellular domain. The extracellular domain of the chimeric immunoreceptor preferably comprises protein sequences from a cancer antigen-specific antibody. Individual T cells of the invention may be CD4⁺/CD8⁻, CD4⁻/CD8⁺, CD4^{-/CD}8⁻ or CD4⁺/CD8⁺. The T cells may be a mixed population of CD4⁺/CD8⁻ and CD4⁻/CD8⁺ cells or a population of a single clone. CD4⁺ T cells of the invention produce helper cytokines (for example IL-2) when co-cultured in vitro with cancer cells. CD8⁺ T cells and some CD4⁺ T cells of the invention lyse cancer target cells in vitro and in vivo.

The cancer-specific immunoreceptor may be specific for any cancer antigen which is useful for recognizing cells of a particular cancer or group of cancers. However in a preferred embodiment, the cancer antigen is CD19. In this embodiment, CD19-specific redirected T cells express CD19-specific chimeric receptor scFvFc:ζ, where scFv designates the V_{H} and V_{L} chains of a single chain monoclonal antibody to CD19, Fc represents at least part of a constant region of a human IgG₁, and ζ represents the intracellular signaling domain of the zeta chain of human CD3. The extracellular domain scFvFc and the intracellular domain are linked by a transmembrane domain such as the transmembrane domain of CD4. The human Fc constant region may be provided by other subclasses of immunoglobulin such as IgG4, for example. See International Patent Application No. PCT/US01/42997, filed 7 Nov. 2001 designating the United States, incorporated herein by reference.

In another preferred embodiment, the cancer antigen is CD20. In this embodiment, CD20-specific redirected T cells express CD20-specific chimeric receptor scFvFc:ζ, where scFv designates the V_{H} and V_{L} chains of a single chain monoclonal antibody to CD20, Fc represents at least part of a constant region of a human IgG₁, and ζ represents the intracellular signaling domain of the zeta chain of human CD3. A transmembrane domain, such as the transmembrane domain of CD4, links the extracellular domain scFvFc with the intracellular domain. The human Fc constant region may be provided by other subclasses of immunoglobulin such as IgG4 for example. See U.S. Pat. No. 6,410,319, incorporated herein by reference.

In a further embodiment, the cancer antigen is found on neuroblastoma and renal carcinoma cells. In this embodiment, neuroblastoma-specific redirected T cells express CE7R-specific chimeric receptor scFvFc:ζ, where scFv designates the V_{H} and V_{L} chains of a single chain monoclonal antibody to CD20, Fc represents at least part of a constant region of a human IgG₁, and ζ represents the intracellular signaling domain of the zeta chain of human CD3. A transmembrane domain, such as the transmembrane domain of CD4, links the extracellular domain scFvFc with the intracellular domain. The human Fc constant region may be provided by other subclasses of immunoglobulin such as IgG4 for example. See U.S. Pat. No. 6,410,319, incorporated herein by reference.

In yet a further embodiment, the cancer antigen is a variant of the IL-13 receptor (IL13R) on glioblastoma cells. In this embodiment, IL13R-specific redirected T cells express IL-13-specific chimeric zetakine receptor IL13:ζ, which fuses a modified IL13 protein in frame with the Fc region, that is at least part of a constant region of a human IgG₁. ζ represents the intracellular signaling domain of the zeta chain of human CD3. A transmembrane domain, such as the transmembrane domain of CD4, links the extracellular domain scFvFc with the intracellular domain. The human Fc constant region may be provided by other subclasses of immunoglobulin such as IgG4 for example. See U.S. Pat. No. 6,410,319, incorporated herein by reference.

In another aspect, the present invention provides a method of treating a cancer in a mammal, which comprises administering bi-specific, cancer antigen-specific redirected T cells to the mammal in a therapeutically effective amount. In one embodiment of this aspect of the invention, a therapeutically effective amount of CD8⁺ bi-specific, cancer antigen-specific redirected T cells are administered to the mammal. The CD8⁺ T cells may be administered in conjunction with CD4⁺ bi-specific, cancer antigen-specific redirected T cells, either simultaneously or sequentially. In a second embodiment of this aspect of the invention, a therapeutically effective amount of CD4⁺ bi-specific, cancer antigen-specific redirected T cells are administered to the mammal. The CD4⁺ bi-specific, cancer antigen-specific redirected T cells may be administered with CD8⁺ bi-specific cytotoxic lymphocytes which express the cancer antigen-specific chimeric receptor cells, either simultaneously or sequentially.

In another aspect, the present invention provides a method of treating a lymphoproliferative disease or autoimmune disease mediated in part by B-cells in a mammal which comprises administering bi-specific, CD19- or CD20-specific redirected T cells to the mammal in a therapeutically effective amount. In one embodiment of this aspect of the invention, a therapeutically effective amount of CD8⁺ bi-specific, CD19- or CD20-specific redirected T cells are administered to the mammal. The CD8⁺ T cells preferably are administered with CD4⁺ bi-specific, CD19- or CD20-specific redirected T cells. In a second embodiment of this aspect of the invention, a therapeutically effective amount of CD4⁺ bi-specific, CD19- or CD20-specific redirected T cells are administered to the mammal. The CD4⁺ bi-specific, CD19- or CD20-specific redirected T cells preferably are administered with CD8⁺ cytotoxic lymphocytes which express the CD19- or CD20-specific chimeric receptor.

In another aspect, the present invention provides a method of vaccinating a mammal with a desired antigen, which comprises administering T cells that have been genetically modified to express a desired antigen. In one embodiment of this aspect of the invention, hygromycin-resistant T cells that express the HyMP1 fusion protein are injected.

In another aspect, the present invention provides a method of treating a cancer in a mammal, which comprises administering bi-specific, cancer antigen-specific redirected T cells to the mammal in a therapeutically effective amount. In one embodiment of this aspect of the invention, a therapeutically effective amount of CD8⁺ bi-specific, cancer antigen-specific redirected T cells are administered to the mammal. The CD8⁺ T cells may be administered with CD4⁺ bi-specific, cancer antigen-specific redirected T cells. In a second embodiment of this aspect of the invention, a therapeutically effective amount of CD4⁺ bi-specific, cancer antigen-specific redirected T cells are administered to the mammal. The CD4⁺ bi-specific, cancer antigen-specific redirected T cells may be administered with CD8⁺ bi-specific cytotoxic lymphocytes which express the cancer antigen-specific chimeric receptor.

To improve the in vivo survival of the adoptively transferred bi-specific T cells selectively, autologous stimulator T cells, that have been genetically modified to express the viral antigen of the bi-specific T cells, are administered as a vaccine. In one embodiment of this aspect of the invention, hygromycin-resistant T cells are injected that express the HyMP1 fusion protein after the MP1- and CD19-bi-specific T cells have been transferred. Judicial use of MP1-presenting stimulator T cells maintains the survival and expands the MP1- and CD19-bi-specific T cells for the purposes of improved MP1- and CD19-specific immunosurveillance and CD19-specific tumor therapy.

In one embodiment of this invention, endogenous influenza-specific human T cells are modified to express a CD19-specific anti-tumor chimeric immunoreceptor as a source of effector cells for adoptive immunotherapy that can be stimulated with influenza antigen in vivo, resulting in the capacity to coordinate cellular anti-leukemia and lymphoma activity in patients with B-lineage malignancies, including those with relapse.

The viral antigen-drug resistance fusion gene results in expression of the viral gene in drug-resistant cells genetically modified to express the fusion gene. This has the following implications:

1. The non-viral electrotransfer of a recombinant protein derived from a viral pathogen avoids potential infection that can be associated with use of whole virus.

2. The viral antigen-drug resistance fusion gene has the potential to present both MHC class I and class II immunologic epitopes derived from the full length of the recombinant viral gene. This has the advantage over the use of virus-derived peptides that require a priori knowledge of the sequence that elicits an immune response for a given CD4 and CD8 T cell in the context of a particular HLA type.

3. Autologous T cells modified with a viral antigen-drug resistance fusion gene can be clinically infused as a vaccine to expand T cells against desired viral epitopes.

4. Autologous T cells modified with a viral antigen-drug resistance fusion gene can be clinically infused as a vaccine strategy to expand tumor-specific T cells that co-express a viral-specific TCR.

5. Autologous T cells modified with the viral antigen-drug resistance fusion gene can be used in vitro to expand T cells against desired viral epitopes.

6. Proteins other than viral genes can be expressed as fusion proteins with hygromycin and drug-resistant autologous T cells genetically modified with these alternative fusion proteins can be used to stimulate desired immune responses in vitro or in vivo (analogous to a vaccine).

The outcome of any treatment preferably is assessed using, for example, flow cytometry or any other convenient method to quantitate the percentage of circulating CD4⁺ and/or CD8⁺MP1-tet⁺ T cells obtained from serial veno-punctures. Additionally, quantitative PCR (Q-PCR) assays using a TaqMan fluorogenic 5′ nuclease reaction also can be used to monitor the in vivo persistence of CD19⁺HyTK⁺ T cell clones. Q-PCR measures the in vivo persistence of CD19-specific genetically modified T cells in mice with a sensitivity approaching 1/100,000 and a specificity approaching 100%.

Anti-tumor response can be determined from, for example, serial measurements of luciferase activity emitted from the genetically modified cells. Histology sections also may be analyzed by immunohistochemistry for co-localization of EGFP⁺ tumor cells and infused bi-specific T cells.

### EXAMPLES

The invention is illustrated by the following examples, which are not intended to limit the invention in any manner. Standard techniques well known in the art or the techniques specifically described therein were utilized.

### Example 1

### Generation of T Cells Expressing MP1 Antigen

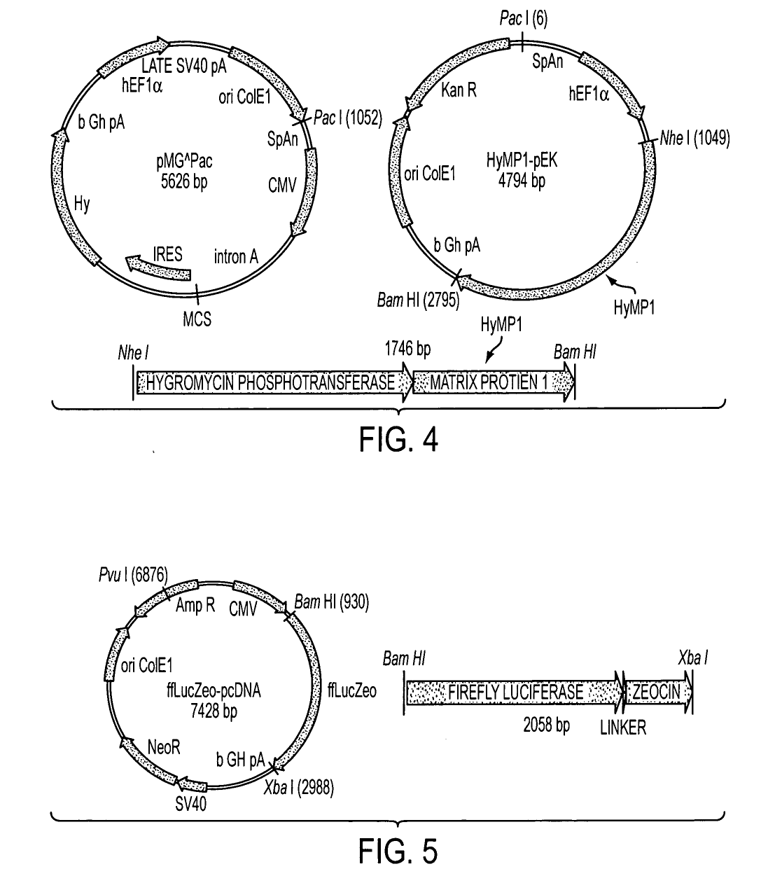

To avoid exposure to infectious virus and circumvent the use of soluble MP1-derived peptide(s), which may not bind to all classical HLA class I antigens, HLA A2⁺ antigen presenting (AP)-T cells were genetically modified by non-viral gene transfer with the DNA plasmid HyMP1-pMG. Hygromycin phosphotransferase (Hy), which confers resistance to the antibiotic hygromycin B in E. coli and mammalian cells, was expressed from the pMG\hat{ }Pac vector. This vector is a modification of the pMG vector (InvivoGen, San Diego, Calif.) by site-directed mutagenesis to remove a Pac I RE site at position 307. See FIG. 4.

The Hy gene plasmid in pMG\hat{ }Pac was changed to Kanamycin/G418-resistance gene to generate the plasmid intermediate pKEN. Subsequent deletion of the neomycin phosphotransferase gene produced the plasmid pEK. This plasmid was used to express the HyMP1 gene, a fusion of a 972 base pair (bp) fragment of the Hy gene from the DNA plasmid pMG cloned with the following PCR primers: 5′-aatactagtgctagcgccgccaccatgaaaaagcctgaactcacc-3′ (5′HyM1; SEQ ID NO:2); 5′-gacctcggttagaagactcatgacttctacacagccatcgg-3′ (HyMP1R; SEQ ID NO:3). A 759 bp fragment of influenza virus A/WSN/33 MP1 gene (GenBank accession number M19374) was cloned with the following PCR primers:

(SEQ ID NO:4)

5′-ccgatggctgtgtagaagtcatgagtcttctaaccgaggtc-3′;
HyMP1F;

(SEQ ID NO:5)

5′-aatggtaccggatcctcacttgaatcgttgcatctgcaccc-3′.
3′HyM1;

Sequencing by the dyedeoxy termination method using (ABI PRISM) dye terminator cycle sequencing ready reaction kit (Perkin Elmer, Foster City, Calif.), according to the manufacturer's instructions, revealed that the MP1 gene differed from the Genbank sequence at amino acid positions 117 and 219 (phenylalanine to leucine and valine to isoleucine, respectively). Based on the HyTK fusion gene sequence, the Hy coding sequence was fused to the 5′ end of MP1 using PCR-splicing by overlap extension (PCR-SOEing) to create a fusion gene with unique 5′ Nhe 1 and 3′ Bam HI restriction enzyme (RE) sites, which wsa used to subclone the fusion gene into pEK to create the plasmid HyMP1-pEK. See FIG. 4. The ffLucZeo fusion gene was cloned by PCR from the plasmid pMOD-LucSh (InvivoGen) with the following primers: 5′-atcggatccgccgccaccatggaggatgccaagaatattaagaaagg-3′ (5′Luc:Zeo; SEQ ID NO:6); 5′-tattctagatcagtcctgctcctctgccacaaagtgc-3′ (SEQ ID NO:7) to introduce a Kosack sequence and unique 5′ Bam HI and 3′ Xba I RE sites which facilitate directional cloning into pcDNA 3.1(+) expression vector, and creating the plasmid ffLucZeo-pcDNA. See FIG. 5. The Pvu I RE site was used to linearize ffLucZeo-pcDNA plasmid before electroporation. Kosack sequences are underlined and start and stop codons are in bold in the oligonucleotide primer sequences above. Correct assembly of HyMP1 and ffLucZeo genes was verified by DNA sequence analyses. Other fusion proteins can be cloned in place of HyMP1, such as Hypp65, a fusion protein of hygromycin phosphotransferase and the CMV tegument protein pp65.

The 1746 bp recombinant fusion protein of hygromycin phosphotransferase and matrix protein 1 (HyMP1) was under control of human elongation factor 1α (hEF1α) hybrid promoter in the plasmid HyMP1-pEK. See FIG. 4. The kanamycin-resistance gene (KanR) was under control of a bacterial promoter (not shown). The Hy gene was under control of human CMV IE promoter and intron. In bacteria, the Hy gene was expressed from the E. coli EM7 promoter (not shown) in pMG\hat{ }Pac. Bovine growth hormone (bGhpA), late SV40 poly A sites (SV40pA), synthetic poly A and pause site (SpAn), E. coli origin of replication (ori ColE1), and some unique RE sites are shown in FIG. 4. The Pac I RE site was used to linearize the plasmids prior to electroporation. This plasmid expresses a fusion gene combining hygromycin phosphotransferase (Hy) and MP1, designated HyMP1.

Lymphoblastoid (LCL) cells, Daudi (CD19⁺) cells, K562 (CD19⁻) cells and primary T cells were maintained in the following medium: RPMI 1640 (Irvine Scientific, Santa Ana, Calif.) supplemented with 2 mM L-Glutamine (Irvine Scientific, Santa Ana, Calif.), 25 mM HEPES (Irvine Scientific), 100 U/mL penicillin, 0.1 mg/mL streptomycin (Irvine Scientific) and 10% heat-inactivated defined fetal calf serum (FCS) (Hyclone, Logan, Utah). U251T (CD19⁻), an HLA A2⁺ adherent tumorgenic line of the human glioma line U251, was maintained in DMEM (Irvine Scientific) supplemented with 10% heat-inactivated FCS, 25 mM HEPES-BSS and 2 mM L-glutamine. Cytocidal concentrations of zeocin (InvivoGen), G418 (CN biosciences, inc, La Jolla, Calif.), and/or hygromycin (Stratagene, Cedar Creek, Tex.) were added to some cultures of Daudi and U251T after non-viral gene transfer.

Primary T cells in the peripheral blood mononuclear cells (PBMC) of healthy volunteers were genetically modified and cultured using methods known in the art. Briefly, 1×10⁶ T-cells from these donors were restimulated every 14 days by adding 30 ng/mL anti-CD3 (OKT3, Ortho Biotech, Raritan, N.J.), 5×10⁷ γ-irradiated PBMC (3,500 cGy) and 1×10⁷ γ-irradiated LCL (8,000 cGy) in RPMI medium. Recombinant human interleukin-2 (rhIL-2) (Chiron, Emeryville, Calif.) at 25 U/mL was added every 48 hours, beginning on day 1 of each 2-week culture cycle. Beginning on day 5 of the cycle, cytocidal concentrations of hygromycin B (0.2 mg/mL) or zeocin (0.2 mg/mL) were added to some T-cell cultures. Between day 10 to 14 of a tissue-culture cycle, some of the T cells were cryopreserved in 10% DMSO and FCS.

To expand MP1-specific T cells, autologous PBMC were co-cultured with γ-irradiated AP-T cells (3,500 cGy) expressing HyMP1 gene at a 1:1 to 5:1 ratio. rhIL-2 at 5 U/mL was added every 48 hours, beginning on day 1 of each 7-day culture cycle. Additional irradiated AP-T cells were added to the culture at a 1:1 or 5:1 ratio every 7 days.

To generate antigen-presenting (AP) cells, T cells were genetically modified with HyMP1-pEK or pMG\hat{ }Pac and expanded in cyctocidal concentrations of hygromycin B. The genetically modified T cells were expanded using 14-day stimulation cycles with OKT3 and IL-2 on a feeder cell layer of irradiated PBMC and LCL in the presence of cytocidal concentrations of hygromycin. Cell lysates along with molecular weight controls were resolved by polyacrylamide gel electrophoresis under reducing conditions. Western blotting with MP1-specific Ab was used to detect the 176 Kda HyMP1.

Western analyses were performed as follows. Twenty million T cells were lysed on ice in 1 ml of RIPA buffer (PBS, 1% NP40, 0.5% sodium deoxycholate, 0.1% SDS) containing 1 tablet/10 ml Complete Protease Inhibitor Cocktail (Boehringer Mannheim, Penzberg, Federal Republic of Germany). After 60 minutes, aliquots of centrifuged supernatant were boiled in an equal volume of loading buffer under reducing conditions and then subjected to SDS-PAGE electrophoresis on precast 12% acrylamide gels (Bio-Rad Laboratories, Hercules, Calif.). Following transfer to nitrocellulose, membranes were blocked for 2 hours in Blotto solution containing 0.07 gm/ml non-fat dried milk. Membranes were washed in T-TBS (0.05% Tween 20 in Tris buffered saline, pH 8.0) and incubated for 2 hours with goat anti-human influenza A MP1 (Immune Systems ltd, Paignton, U.K.). After washing in T-TBS, the membranes were incubated for 1 hour with a 1:500 dilution of alkaline phosphatase-conjugated mouse antibody specific for goat IgG. The membranes were rinsed in T-TBS and then developed with 30 ml of AKP solution (Promega, Madison, Wis.) according to manufacturer's instructions. The chemiluminescence was measured over a 2-hour period.

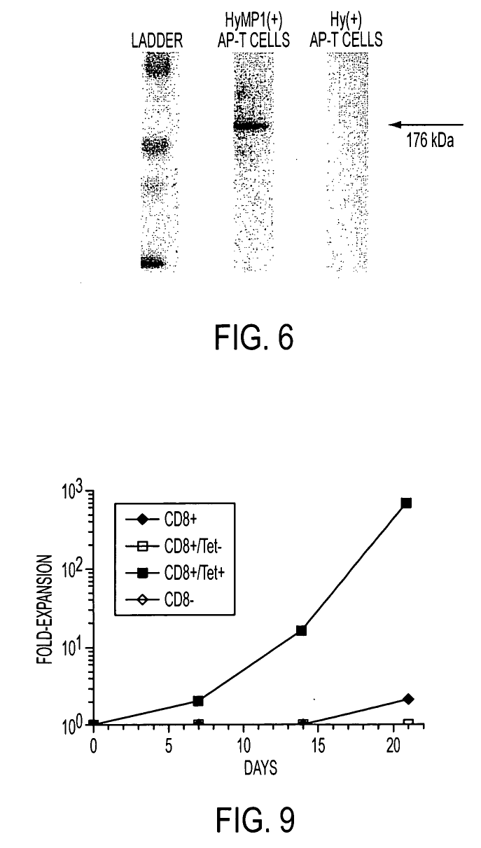

Western blot analysis showed that hygromycin-resistant T cells expressed recombinant MP1 (expected MW 176 Kda). See FIG. 6. The protein was not present in control HLA A2+T cells modified with pMG\hat{ }Pac plasmid to express the Hy gene alone.

For non-viral gene transfer, two micrograms of linearized DNA plasmid pCI-ΔCD19, which expresses truncated CD19 (lacking the cytoplasmic domain) in the plasmid pCI-neo (Promega, Madison, Wis.), or 2 μg HyMP1-pEK, or 2 μg pMG\hat{ }Pac was premixed in lipofectamine and gently dispersed onto U251T cells expanding at log-phase growth in 6-well tissue culture plates. After 72 hours, the cells were grown in cytocidal concentrations of G418 (0.25 mg/mL) or hygromycin (0.2 mg/mL), respectively. To produce cells expressing both CD19 antigen and MP1, the CD19⁺ U251T cells were retransfected with HyMP1-pEK plasmid and grown on cytocidal concentrations of both G418 and hygromycin. Transfection of 400 μL of 8×10⁶ Daudi cells was achieved using a single pulse of 240 V for 40 μsec in a Multiporator device (Eppendorf AG Hamburg, Germany) with 10 μg linearized plasmid ffLucZeo-pcDNA in hypo-osmolar buffer. Beginning three days after electroporation, cytocidal concentrations of G418 (1.4 mg/mL) were added. Transfection of 400 μL of 8×10⁶/mL primary human T cells was achieved three days after stimulation with 30 ng/mL of OKT3 by electroporating with a single pulse of 250 V for 40 μsec using a Multiporator device with 10 μg of linearized DNA plasmid in hypo-osmolar buffer. Beginning two days after electroporation, cytocidal concentrations of hygromycin B (0.2 mg/mL) were added.

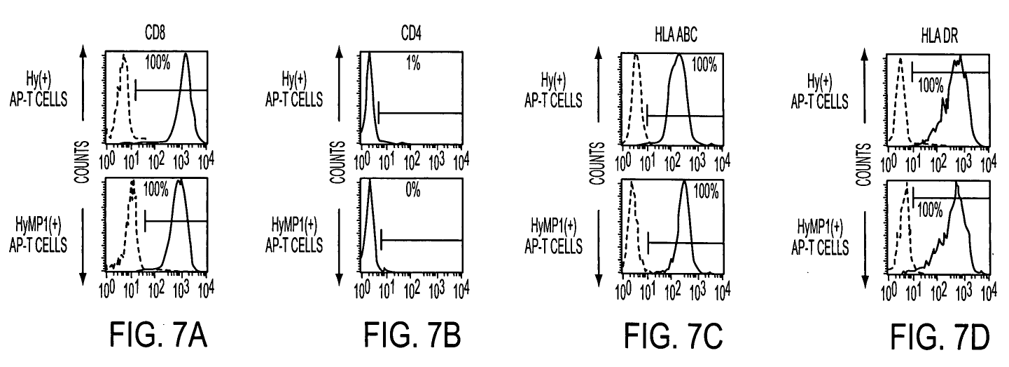

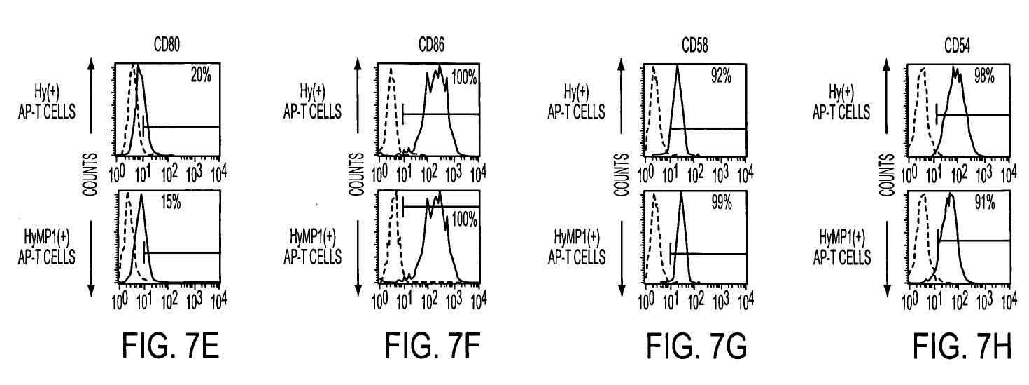

Induction of a proper adaptive immune response is dependent on the correct transfer of information between APCs and antigen-specific CD8⁺ T cells. Communication between the cells depends on expression of classical HLA class I molecules that can be augmented by T-cell activation molecules. The AP T-cell lines, expanded by repetitive OKT3-stimulation in the presence of cytocidal concentrations of hygromycin B, were characterized by flow cytometry to determine their status: CD8⁺, CD4⁻, MHC class I⁺ and class II⁺, CD54⁺ (ICAM-1), CD58⁺ (LFA-3), CD80^{dim}, CD83⁻, CD86⁺, 41BBL⁻, and not bound by NKG2D-Fc. See FIG. 7.

Flow cytometry was performed as follows. Combinations of some of the following fluorescein isothiocyanate (FITC)-, phycoerythrin (PE)-, or CyChrome-conjugated reagents were used for staining prior to cell sorting: Annexin V, anti-TCRαβ, anti-CD3, anti-CD8, anti-CD4, anti-CD10, anti-CD19, anti-CD28, anti-CD45, anti-CD80, anti-CD86, anti-CD54, anti-CD58, anti-HLA ABC, anti-HLA DR and anti-NKG2D (BD Biosciences). In some assays FITC-conjuagted goat anti-human Fc (Jackson Immunoresearch) at 1/20 dilution was used to detect cell surface expression of CD19R. In some cases, PE-conjugated MP1-tetramer was used. This reagent recognizes human CD8⁺ T cells specific for the glycineisoleucine-leucine-glycine-phenylalanine-valine-phenylalaninethreonine-leucine peptide (GILGFVFTL; SEQ ID NO:1) from influenza MP1 in combination with the HLA-A*0201 allele (Beckman Coulter Immunomics Operations, San Diego, Calif.). Some experiments used biotin-conjugated mAb specific for TCR Vβ17 and CyChrome-conjugated streptavidin. In some experiments, CyChrome-conjugated mAbs were replaced with 1 μg/mL propidium iodide (PI), which was used to exclude non-viable cells from analysis. Data was acquired on a FACScan (BD Biosciences) and the percentage of cells in a region of analysis was calculated using CellQuest version 3.3 (BD Biosciences). Fluorescence activated cell sorting using a MoFlo MLS (Dako-Cytomation, Fort Collins, Co) was used to isolate T cells bound by MP1-tetramer.

FIG. 7 provides histograms showing binding of specific mAbs (FIG. 7, bold line), relative to isotype control (FIG. 7, dotted line), for AP-T cells genetically modified with pMG\hat{ }Pac or HyMP1-pEK. The relative percentage of cells in each gate is indicated. The AP-T cells are capable of presenting antigen through MHC class I and using at least some known co-stimulatory molecules to augment cellular interaction.

### Example 2

### In Vitro T-Cell Culture System to Expand MP1-Specific CD8⁺ T Cells Using Autologous T Cells Presenting MP1

A kinetic study determined whether the HyMP1-expressing, genetically modified AP-T cells could directly stimulate expansion of CD8⁺ MP1-specific T cells in vitro. During three weeks of co-culture with irradiated autologous AP-T cells expressing the HyMP1 gene, flow cytometry was used to demonstrate the expansion of MP1-tetramer⁺ T cells from a HLA A2⁺ healthy volunteer donor. HLA A2+PBMC were co-cultured for 21 days in the presence of low-dose IL-2 (A) without the addition of autologous AP-T cells, or with a 5:1 (Responder:Stimulator) T-cell ratio of γ-irradiated hygromycin-resistant (B) Hy⁺ AP-T cells (that do not express MP1), or (C) γ-irradiated HyMP1⁺AP-T cells. AP-T cells were re-added to the culture system every 7 days. Binding of a control CMV pp65-tetramer on day 21 was negligible. Dead cells were excluded from analysis upon uptake of propidium iodide (PI).

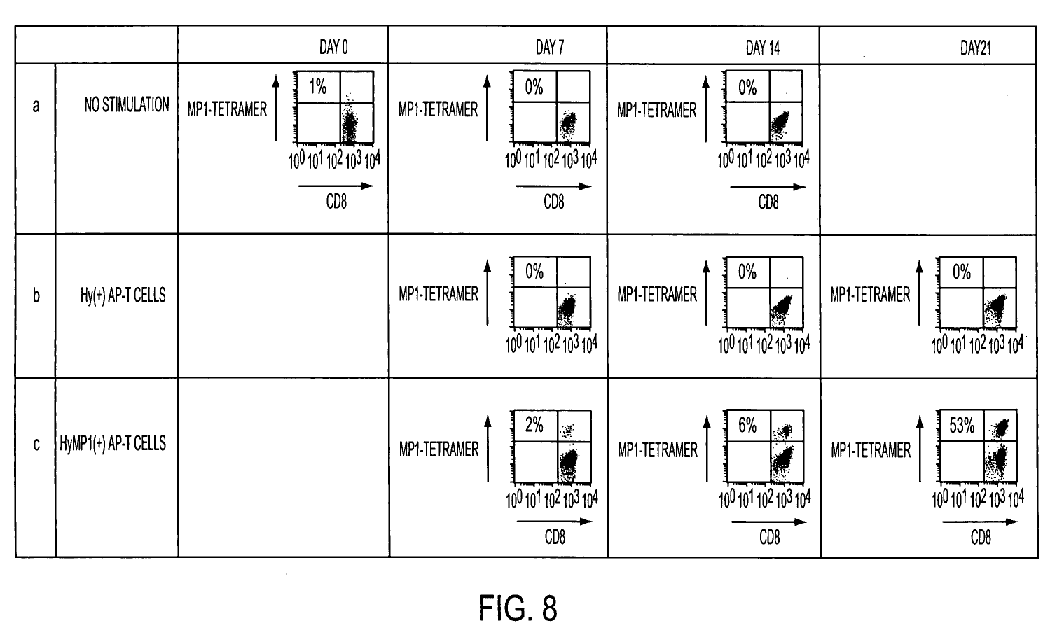

The binding of MP1-tetramer to CD8⁺ T cells was measured by multiparameter flow cytometry every 7 days, prior to the addition of the stimulator AP-T cells, and is reported as a percentage of CD8⁺ T cells. See FIG. 8. Dead cells were excluded from analysis upon taking up PI. The AP-T cells are not bound by MP1-tetramer. HLA A2⁺ HyMP1⁺ and Hy⁺ AP-T cells are not bound by MP1-tetramer.

The percentage of MP1-tetramer⁺ CD8⁺ T cells rapidly increased from 1% (pre-stimulation) to 50% after 21 days of co-culture. By 7 days of stimulation, the percentage of MP1-tetramer⁺ T cells was 2%, which compares favorably with the expansion of MP1 tetramer⁺ T cells cultured on mature dendritic cells (DCs) infected with live influenza virus.

To control for the specificity of the T-cell expansion process, HLA-A2⁺ T cells were co-cultured under identical conditions without AP-T cells or with AP-T cells expressing hygromycin but not MP1. One million HLA A2⁺ PBMC were co-cultured for 21 days at a 5:1 (Responder:Stimulator) T-cell ratio in low-dose rhIL-2 with thawed γ-irradiated autologous HyMP1⁺ AP-T cells. Fresh AP-T cells were added every 7 days. Viable cells were counted by the trypan blue dye exclusion method. There was no expansion of MP1-tetramer⁺ T cells. See FIG. 9. In addition, pp65-tetramer⁺ T cells from a CMV-seropositive individual did not expand when co-cultured with MP1⁺ AP-T cells. Enumeration studies demonstrated that viable MP1-tetramer⁺ CD8⁺ T cells increased in number up to 630-fold over the 3-week culturing period with MP1⁺ AP-T cells.

CD8⁺ MP1-specific memory T cells are known to expand on tetramer-identified mature DCs infected with influenza, correlated with the ability to secrete interferon-γ (IFN-γ) in response to MP1-antigen. Therefore, to demonstrate that MP1+AP-T cells could expand to form functional MP1-specific T cells, MP1-tetramer⁺ T cells were isolated by flow cytometry sorting and assayed for T_{c}1 cytokines produced upon co-culture with irradiated autologous AP-T cells. The following methods were used for analysis of cytokine production. One million T-cell responder cells were co-cultured at a 1:1 ratio in 12-well tissue culture plates with γ-irradiated U251T (8,000 cGy), Daudi (8,000 cGy), and/or AP-T cells (3,500 cGy) in 2 mL RPMI medium as described above. After a 48-hour incubation at 37° C., the conditioned medium was assayed by cytometric bead array (CBA) using the (BD Pharmingen) Human Th1/Th2 Cytokine kit according to the manufacturer's instructions using a FACScan instrument equipped with an automated 96-well plate reader. Cytokine concentrations then were calculated.

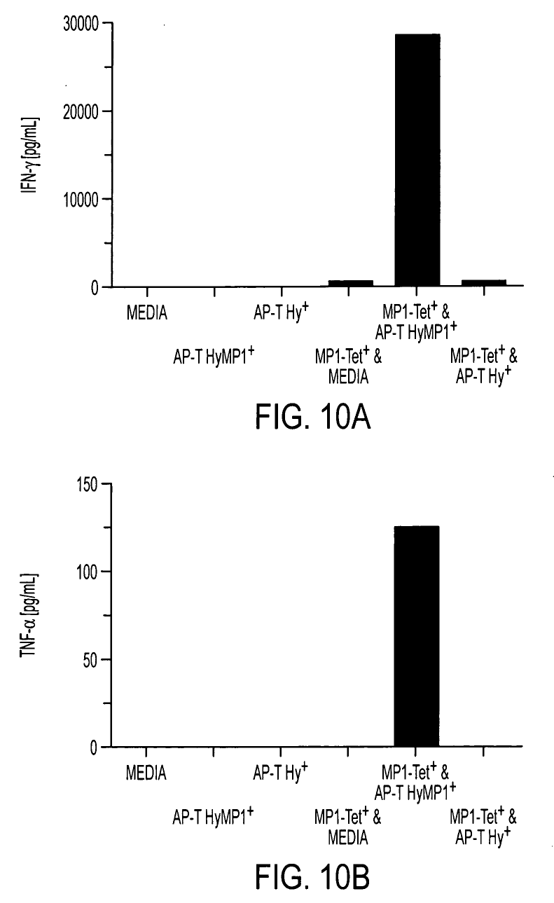

The MP1-tetramer⁺ T cells produced increased IFN-γ (11-fold) and tumor necrosis factor-alpha (TNF-α; 7-fold) over incubating MP1-specific T cells incubated in media alone or with autologous Hy⁺ AP-T cells that do not express MP1. See FIG. 10. Under these control culture conditions there was no detectable IL-2 produced by stimulation through the endogenous MP1-specific αβTCR, consistent with the phenotype of a type 1 CD8⁺ CD28⁻ effector T cell that had no detectable autocrine IL-2 signaling ability. To confirm that the T-cell population receiving the activation signal to release cytokine was the effector cells, production of IFN-γ and TNF-α was measured. There was no detectable production of these cytokines from these irradiated AP-T cells.

### Example 3

### MP1-Specific T Cells can be Genetically Modified to Express a CD19-Specific Chimeric Immunoreceptor

To determine if MP1-specific T cells could be rendered specific for CD19, the CD19R gene was introduced into MP1-tetramer⁺ T cells. This genetic modification of T cells was accomplished using non-viral electrotransfer of a DNA expression plasmid designated CD19R/HyTK-pMG which codes for both CD19R and a bifunctional fusion gene that combines hygromycin phosphotransferase and herpes virus thymidine kinase (HyTK). The specificity of CD19R is derived from the variable regions of a mouse monoclonal antibody (mAb) specific for CD19, tethered to the T cell via a modified human IgG4 hinge and Fc-fragment attached to the human CD4 transmembrane domain. Upon binding CD19, the genetically modified T cells are activated via the cytoplasmic CD3-ζ chain attached to the chimeric immunoreceptor.

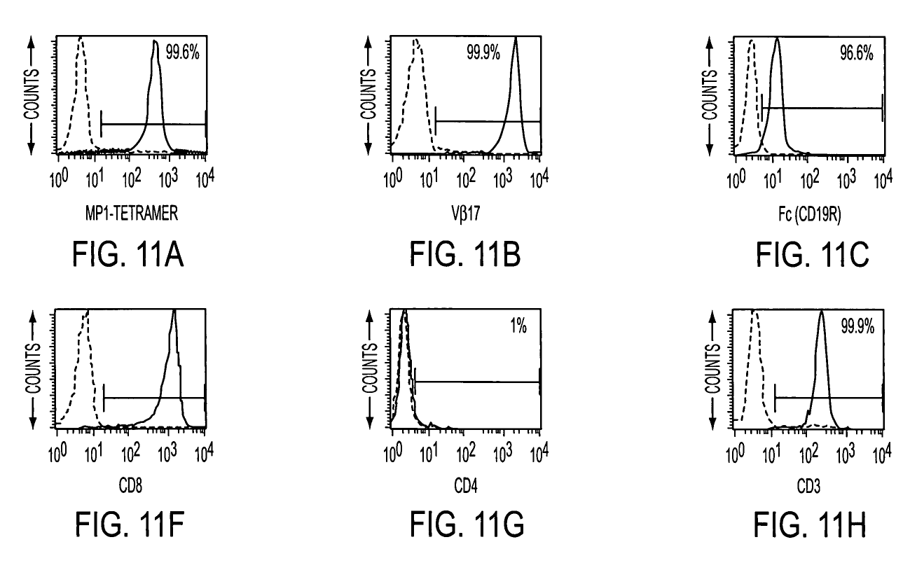

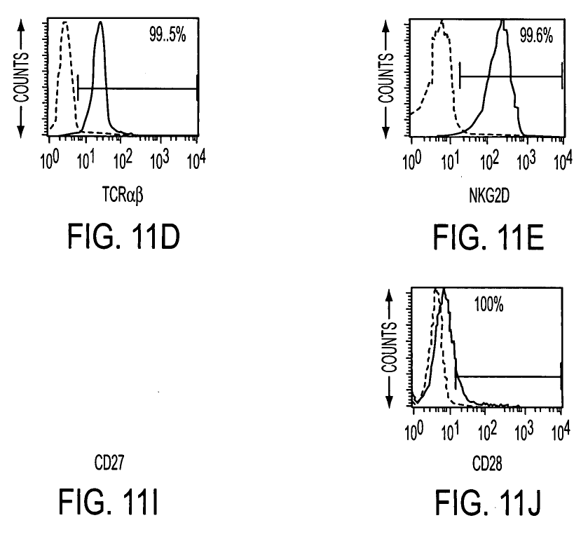

HLA A2⁺ T cells were expanded on autologous HyMP1⁺ AP T cells, FACS sorted for binding to MP1-tetramer, genetically modified with CD19R/HyTK-pMG. After numeric expansion of the genetically modified cells in vitro using 14-day stimulation cycles with OKT3 and IL-2 on a feeder cell layer of irradiated PBMC and LCL in the presence of cytocidal concentrations of hygromycin, flow cytometry analyses demonstrated that these HLA A2⁺ T cells remained MP1-tetramer⁺ and were also TCR Vβ17⁺. See FIG. 11.

The presence at the cell surface of the introduced chimeric immunoreceptor, which includes C_{H}2 and C_{H}3 immunoglobulin domains, was documented by flow cytometry. Ninety-six percent of the expanded hygromycin-resistant MP1-tetramer⁺ CTL were Fc⁺. See FIG. 11. This is consistent with the finding that the TCR Vβ17 is the dominant Vβ segment used by HLA-A2-restricted CTL that recognize MP1₅₈₋₆₆. Furthermore, Western blot of reduced whole T-cell lysates probed using a mAb specific for CD3-ζ chain demonstrated that the MP1-tetramer⁺Fc⁺ T cells expressed a 66-kDa protein consistent with the expected size of the introduced chimeric zeta chain.

Since the ability of T cells to achieve full activation after stimulation through αβTCR is dependent on co-expression of T-cell co-stimulatory molecules, flow cytometry was used also to characterize the phenotype of the expanded MP1-tetramer⁺Fc⁺ effector T-cell population. The cells were confirmed to be CD8⁺, CD4⁻, TCRαβ⁺, CD3⁺, CD27⁻, CD28⁻, CD54⁺, CD58⁺, CD137⁻ (41BB). See FIG. 11.

### Example 4

### Endogenous αβTCR and Introduced CD19-Specific Chimeric Immunoreceptor Co-Cap in Response to MP1 and CD19 Antigens

Formation of an immunological synapse between effector T cells and target cells generates the recognition signals for T-cell activation. This synapse begins with clustering of receptors docking with antigen and leads to the centralized accumulation of TCRs and receptor capping. This receptor capping is microscopically visible using fluorescently-labeled Abs.

To induce capping, 10⁶ HLA A2⁺ MP1-tetramer⁺Fc⁺ T cells were co-cultured with HLA A2⁺CD19⁺MP1⁺ U251T cells at 37° C. for 60 minutes. T-cell media containing 0.2% azide was then added to the cells to stop the capping event. The cells then were fixed using 1 ml PBS containing 4% formaldehyde for 20 minutes at 4° C. and afterwards washed and stained with FITC-conjugated goat antibody specific for human Fc to detect CD19R. After washing, the cells were stained with PE-conjugated anti-CD49c and biotin-conjugated anti-Vβ17 followed by CyChrome-conjugated strepatavidin. The cells were resuspended in PBS containing 0.5% formaldehyde and collected using the ImageStream 100™ (IS100™, Amnis Corporation, Seattle Wash.) imaging flow cytometer. The IS100™ instrument uses an arc illumination source for brightfield imagery and a 488 nm laser for fluorescence excitation. The instrument was configured to collect five spectrally decomposed images of each cell in flow (brightfield, laser scatter, FITC, PE, and CyChrome. A data set of 20,000 cells was analyzed using the IDEAS™ image analysis software to create scatter plots and view image galleries. Events that were positive for both CD49cPE and Vβ17 CyChrome were isolated and scrutinized for both conjugate formation and the presence of Fc FITC capping.

Because the APC cells exhibited a high level of autofluorescence in the FITC channel, candidate events identified using the bivariate histograms were gated into a discrete image gallery and reviewed individually to find capping of Vβ17 and Fc. Whether CD8⁺ T cells expressing CD19R could continue to cap endogenous αβTCR and acquire an ability to cap the introduced chimeric immunoreceptor was investigated using this technique.

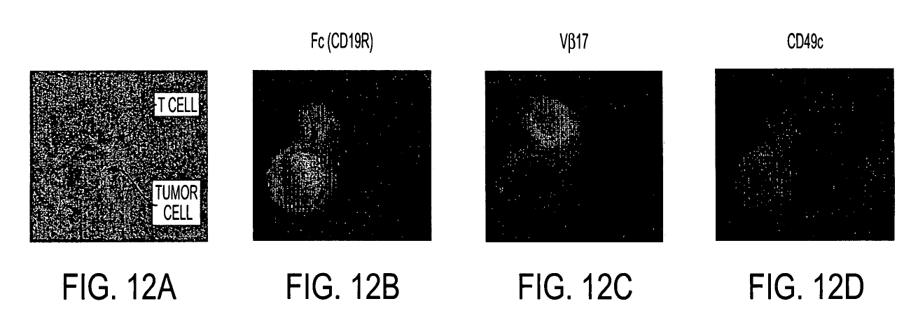

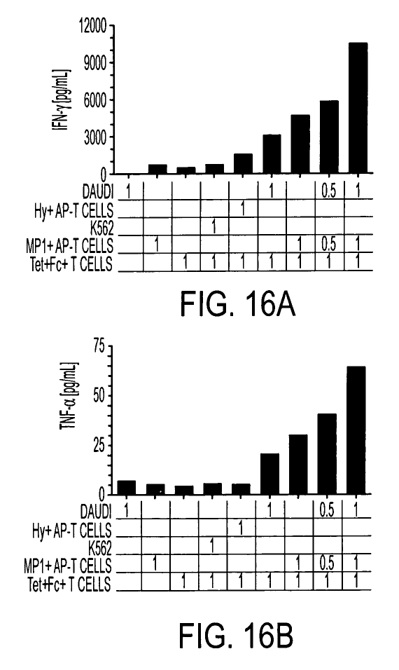

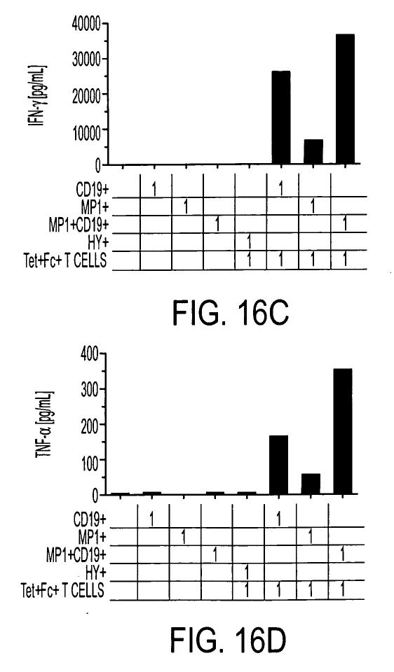

The MP1-tetramer⁺Fc^{+ CD}8⁺ T cells, which express the endogenous Vβ17⁺ TCR and the introduced CD19R gene, were co-cultured with HLA A2⁺ U251T target tumor cells that had been genetically modified with the plasmids pCI-ΔCD19 and HyMP1-pMG, to co-express CD19 and MP1. Using a combination of high-speed microscopy with multiparameter flow cytometry both the chimeric immunoreceptor and the endogenous TCR were demonstrated to respond to a polarizing stimulus, indicating that the MP1-tetramer⁺Fc⁺T cells could be independently and simultaneously activated through either receptor. See FIG. 12. T cells and tumor cells that were docked together, as identified by (12A) bright field image, were analyzed for capping of (12B) endogenous αβTCR, with biotinylated mAb specific for Vβ17, and (12C) introduced CD19-specific chimeric immunoreceptor with FITC-conjugated anti-Fc using the IS100™. Tumor cells were identified by binding of PE-conjugated anti-CD49c, a monoclonal antibody that recognizes an α3 integrin on U251T cells. Conjugate events were approximately 30 μm and imaged with a 0.75 objective at 0.5 μm pixel resolution on the IS100™. The phenotype of the genetically modified U251T cells is discussed below in the context of FIG. 16.

### Example 5

### MP1-Tetramer⁺Fc⁺ T Cells are Functionally Bi-Specific

A 4-hour CRA determined whether the MP1-tetramer⁺Fc⁺ CD8⁺ T cells could be activated for lysis though both the endogenous and the introduced immunoreceptor. The general procedure for CRAs was as follows. The cytolytic activity of effector (E) T cells was determined by chromium release assay (CRA) using triplicate V-bottom wells in a 96-well plate (Costar, Cambridge, Mass.) containing Na⁵¹CrO₄-labeled Daudi, U251T, AP-T cells, primary ALL blasts, or K562 target (T) cells according to methods known in the art. The effector T cells were harvested 10-14 days after stimulation with OKT3, washed, and then incubated with 5×10³ target cells in triplicate. After centrifugation and incubation at 37° C. for 4 hours, aliquots of cell-free supernatant were harvested and counted. The percent specific cytolysis was calculated from the release of ⁵¹Cr as follows: [(experimental ⁵¹Cr)−(control ⁵¹Cr)]/[(maximal ⁵¹Cr)−(control ⁵¹Cr)]×100. Control wells contained target cells incubated in media. Maximal ⁵¹Cr was determined by measuring the ⁵¹Cr content released by target cells lysed with 2% SDS. Data are reported as an average.

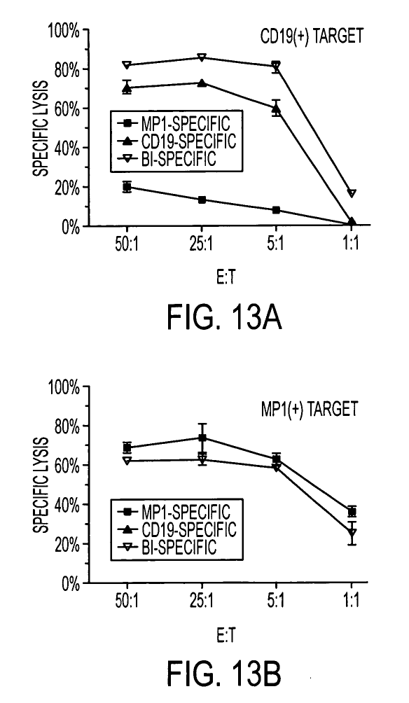

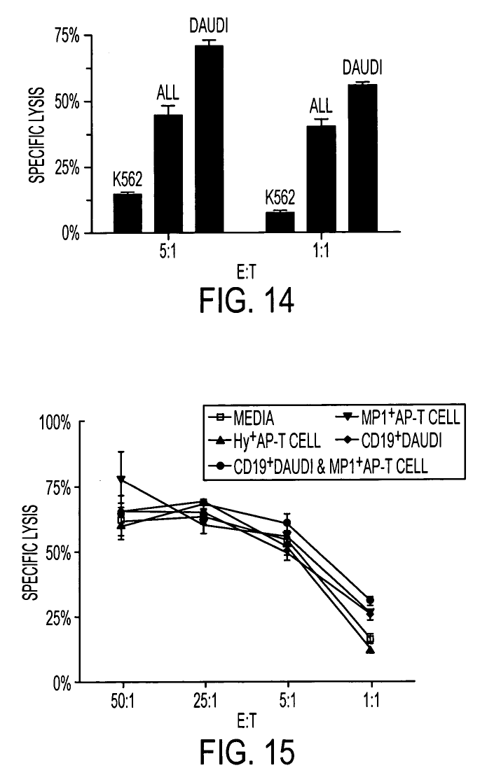

⁵¹Cr-labeled targets CD19⁺ Daudi cells (FIG. 13) or MP1⁺ HLA A2⁺ AP-T cells (FIG. 14) were incubated with CD19-specific T cells, HLA A2⁺ MP1-specific T cells, or HLA A2⁺ MP1- and CD19-bi-specific T cells. The mean and standard deviation specific lysis was calculated after 4 hours. The MP1-tetramer⁺Fc⁺ T cells were able to lyse both CD19⁺ and MP1⁺ targets. In contrast, a T-cell clone expressing only CD19R could lyse only the CD19+target and the MP1-tetramer⁺ T cells could lyse only the MP1⁺ target. See FIG. 13.

Because the MP1-tetramer⁺Fc⁺ T cells are designed for use in the clinic, it was desirable to confirm that these effector T cells could recognize primary B-lineage ALL cells. To this end, ⁵¹Cr-labeled blasts were incubated with MP1- and CD19-bi-specific T cells. See FIG. 14. The mean and standard deviation specific lysis was calculated after 4 hours. The ALL blasts (CD19⁺CD10⁺CD45⁻) represented 56% of the total population and 78% of the lymphoid-gated population. The data in FIG. 14 demonstrate this recognition and are consistent with the genetically modified T cells being bi-specific.

### Example 6

### MP1-Tetramer⁺Fc⁺ T Cells Retain Specificity for CD19⁺ Tumor After Interacting with MP1 and CD19 Antigens

Since CTL have a propensity to undergo activation-induced cell death (AICD) upon restimulation, loss of function is a potential consequence of simultaneous signaling through both endogenous and introduced immunoreceptors. If the MP1-tetramer⁺Fc⁺ T cells are to be useful in a clinical environment, they preferably remain able to target CD19⁺ tumor after stimulation through the endogenous αβTCR with MP1 antigen. To model this behavior in vitro in using a method which correlates to in vivo results, the bi-specific effector cells were pre-exposed to stimulator AP-T cells and/or tumor cells expressing a combination of MP1 and CD19 antigens.

As shown in FIG. 14, MP1-tetramer⁺Fc⁺ T cells can lyse CD19⁺ target cells after prior exposure to MP1⁺ and/or CD19⁺ target cells. HLA A2⁺ MP1- and CD19-bi-specific T cells were incubated at 37° C. in media, or at a 1:1 ratio with autologous Hy⁺ AP-T cells, MP1⁺ AP-T cells, CD19⁺ Daudi cells, or a 1:1 mixture of MP1⁺ AP-T cells and CD19⁺ Daudi cells. After 5 days of exposure, a 4-hour CRA revealed no apparent loss of lytic activity of the MP1-tetramer⁺Fc⁺ T cells for CD19⁺ Daudi cells despite prior exposure to MP1 and/or CD19 antigens, compared with the same effector cells incubated in media alone. See FIG. 15. Lysis of CD19-K562 cells under these conditions at E:T of 25:1 was 6-13%. These data demonstrate that the bi-specific T cells remain cytolytic, even after activation through the endogenous and/or chimeric immunoreceptors.

### Example 7

### MP1-Tetramer⁺Fc⁺ T Cells can Achieve Supra-Physiologic Activation for Cytokine Release After Interacting with MP1 and CD19 Antigens

To investigate whether MP1-tetramer⁺Fc⁺ T cells expressing two functional immunoreceptors are capable of simultaneous signaling through each immunoreceptor which leads to supra-physiologic activation, the ability of the MP1-tetramer⁺Fc⁺ effector T cells to be activated for cytokine secretion was determined by culturing the effector cells with stimulator cells expressing CD19 or MP1 antigen. See FIG. 16.

For FIG. 16A, HLA A2⁺MP1- and CD19-bi-specific T cells were incubated at 37° C. with γ-irradiated CD19⁻ K562 cells, or autologous Hy⁺ AP-T cells, HyMP1⁺ AP-T cells, CD19⁺ Daudi cells, or 1:1 mixture of MP1⁺ AP-T cells and CD19⁺ Daudi cells. After 48 hours of culture, assays detected a 5 to 8-fold increase in TNFα and IFN-γ when co-cultured with CD19⁺ Daudi, and a 7 to 12-fold increase when co-cultured with MP1⁺ AP-T cells, compared to control cultures (effector cells cultured in the absence of stimulator cells). The low background level of cytokine released from both target cells in the absence of MP1-tetramer⁺Fc⁺ T cells and effector cells cultured with CD19⁻ K562 cells or Hy⁺ AP-T cells ensured that the cytokine produced was specific for the introduced and endogenous immunoreceptor contacting their respective antigen. These data confirm that the MP1-tetramer⁺Fc⁺ T cells are activated in response to either CD19 or MP1 antigens.

To investigate whether exposure of MP1-tetramer⁺Fc⁺ T cells to both CD19 and MP1 antigens resulted in augmented cytokine production the responder, T cells were co-cultured with a mixture of MP1⁺ AP-T cells and CD19⁺ Daudi cells at a 1:1:1 ratio. HLA A2⁺ MP1- and CD19-bi-specific T cells were incubated at 37° C. in media, or with mitomycin C-treated HLA A2⁺ U251T cells, genetically modified with plasmids pMG\hat{ }Pac, pCI-ΔCD19, and/or HyMP1-pEK. Flow cytometry data using anti-CD19 mAb demonstrated that 90% of the parental and MP1⁺ U251T cells modified with the plasmid pCI-ΔCD19 expressed CD19 with a median fluorescent intensity similar to Daudi cells. RT-PCR analyses using MP1-specific primers, spanning an intron in the expression plasmid, were used to demonstrate that the parental and CD19⁺ U251T cells modified with the plasmid HyMP1-pMG expressed MP1. U251T cells modified with the plasmid pMG\hat{ }Pac did not.

After 48-hours, the concentration of IFN-γ and TNF-α was determined using a CBA. Relative ratios of responding T cells and stimulator cells are shown in the Figure. This co-culture resulted in a 200-300% increase in produced IFN-γ and TNF-α, compared with the levels of these cytokines produced when the effector cells were incubated individually with the AP-T and Daudi cell targets. The increased cytokine production persisted even when the relative numbers of MP1⁺ AP-T cells and Daudi cells simultaneously cultured with the effector cells was reduced by half.

Since the presentation of MP1 and CD19 antigens was sequential (as these antigens were expressed by different cells), whether augmented cytokine production could be achieved when MP1 tetramer⁺ Fc⁺ T cells dock with stimulator cells presenting both antigens also was investigated. This was accomplished using HLA A2⁺ U251T cells that had been genetically modified to express truncated CD19 (so as to not interfere with cell growth) and MP1, or CD19 and MP1. To control for specificity of cytokine release, U251T cells also were genetically modified with the plasmid pMG\hat{ }Pac to express Hy gene, but not CD19 nor MP1. After 48 hours of co-culture with CD19⁺MP1⁺ U251T cells, the responding MP1 tetramer⁺Fc⁺ T cells released 500-600% more IFN-γ and TNF-α, compared with co-culture with MP1⁺ U251T cells, and 100-200% more IFN-γ and TNF-α compared with co-culture with CD19⁺ U251T cells. See FIG. 16. The MP1-tetramer⁺Fc⁺ T cells produced more T_{c}1 cytokines upon co-culture with CD19⁺ U251T stimulator cells, compared with MP1⁺ U251T cells, which may be due to a relative lack of processing and presentation of the MP1. Nevertheless, stimulator cells that simultaneously present MP1 and CD19 antigens activate MP1-tetramer⁺Fc⁺ T cells for enhanced cytokine production.

### Example 8

### Proliferation of MP1-Tetramer⁺Fc⁺ T Cells is Augmented when Both MP1 and CD19 Antigens are Present

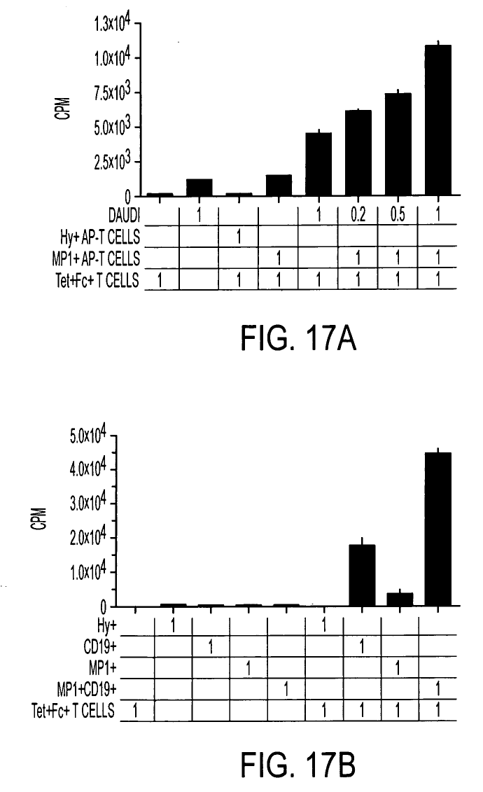

Stimulation through the endogenous αβTCR can activate T cells for proliferation, whereas direct activation of human T cells via chimeric CD3-ζ, such as via chimeric immunoreceptors specific for G_{D2} or CD33, apparently are not sufficient to induce proliferation. Therefore, the replicative capacity of the MP1-tetramer⁺Fc⁺ T cells upon exposure to MP1 and/or CD19 antigens was evaluated. See FIG. 17.

Methods for T cell proliferation were as follows. Five thousand T-cell responders were co-cultured in quadruplicate in 96-well U-bottom plates at a 1:1 ratio with U251T stimulator cells (pretreated 48-hours prior to co-culture for 45 minutes with 50 μg/mL of mitomycin-C (Sigma-Aldrich, St. Louis, Mich.), or γ-irradiated (3,500 cGy) AP-T cells. After the 48 hour incubation, the wells were pulsed with 1 μCi/well [methyl-³H]-thymidine (ICN Biochemicals Inc., Cleveland, Ohio). Twelve hours later, DNA was harvested and ³H-TdR incorporation was counted with a liquid scintillation β-counter (Beckman Coulter Scintillation Counter LS 6500, Fullerton, Calif., or TopCount NXT). Data are reported as the mean±the standard deviation.

First, HLA A2⁺ MP1- and CD19-bi-specific T cells were incubated at 37° C. in media, or with autologous Hy⁺ AP-T cells, HyMP1⁺ AP-T cells, CD19⁺ Daudi cells, or mixtures of MP1+AP-T cells and CD19+Daudi cells. See FIG. 17A. Stimulation through the endogenous immunoreceptor resulted in a greater increase in ³H-thymidine incorporation upon co-culture of the effector cells with MP1⁺ AP-T cells or MP1⁺ U251T cells, respectively, compared with culture of the responder T cells in media or Hy⁺ AP-T cells or Hy⁺ U251T cells (control). Second, HLA A2⁺MP1- and CD19-bi-specific T cells were incubated at 37° C. in media, or with HLA A2⁺ U251T cells genetically modified with plasmids pMG\hat{ }Pac, pCI-ΔCD19, and/or HyMP1-pEK. See FIG. 17B. The relative ratio of responder T cells to mitomycin C-treated or γ-irradiated stimulator cells is shown in the Figures. Proliferation after 72 hours was determined and reported as mean±standard deviation.

These data indicate that MP1 tetramer⁺Fc⁺ Tcells proliferate in response to either MP1 or CD19 antigens. However, there were differences in the relative proliferative potential upon activation through the αβTCR compared with CD19R. For instance, the relative proliferation of MP1-tetramer⁺Fc⁺ T cells responding to CD19⁺ U251T cells was greater than for MP1⁺ U251T cells, which was the same relative order as for cytokine production and may be due to relative differences in antigen density due to a lack of processing and presentation of MP1 by U251T cells.

The potential for supra-physiologic activation of T cells was examined by determining the ability of MP1-tetramer⁺Fc⁺ T cells to proliferate when sequentially or simultaneously exposed to both CD19 and MP1 antigens. This was accomplished by co-culturing the responding T cells with mixtures of CD19⁺ Daudi and MP1⁺ AP-T cells and co-culturing the responding T cells with CD19⁺MP1⁺ U251T cells. When both CD19 and MP1 antigens were present, the MP1-tetramer⁺Fc⁺ T cells demonstrated increased proliferation compared with incubating the responding T cells with either antigen alone. See FIG. 17.

Other data indicate that an explanation for this relative increase in proliferation is a relative reduction in antigen-dependent apoptosis when MP1-tetramer⁺Fc⁺ T cells dock with two antigens. These data are consistent with the data respecting cytokines and indicate that contact with both CD19 and MP1 antigens results in augmented T-cell activation. In addition, these data confirm the usefulness of these methods in vivo, since the bi-specific MP1-tetramer⁺Fc⁺ T cells can proliferate in response to MP1-antigen despite the anticipated presence of abundant CD19 antigen on normal and malignant tissue.

### Example 9

### Development of AP-T Cells for Use In Vivo

The biologic half-life of these human T cells when adoptively transferred is a relevant factor when using AP-T cells as a T-cell vaccine. To test this parameter, HLA A2⁺ T cells, genetically modified with the vector ffLuc/neo-pMG to express the ffLuc reporter gene, were introduced into the peritoneum of NOD/scid mice. See FIG. 5, which is a schematic drawing of a plasmid expressing ffLucZeo.

The fusion protein of firefly (Photinus pyralis) luciferase (ffLuc) reporter gene and zeocin-resistance gene is under control of the human CMV promoter. The ampicillin-resistance gene (AmpR) is under control of a bacterial promoter (not shown). The bovine growth hormone (bGhpA), E. coli origin of replication, and some unique RE sites are shown. The Pvu I RE site was used to linearized the plasmid prior to electroporation.

Relative luciferase activity from 10⁶ transfected and non-transfected cells was determined. Firefly luciferase gene activities were measured from 10⁶ cells using the Luciferase Assay System (Promega) according to the manufacturer's protocol. Measurements were performed in triplicate using a LS 6500 Scintillation Counter (Beckman Coulter) and results are reported as mean±standard deviation.

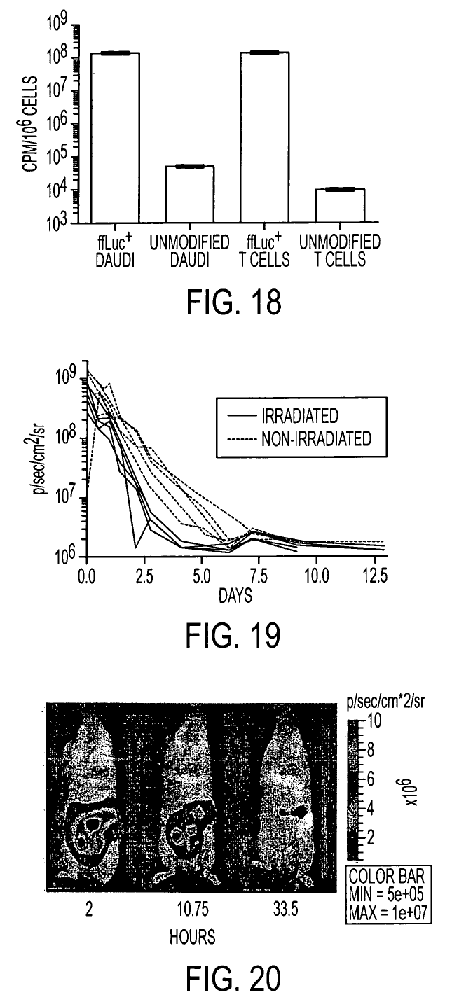

The data are reported in FIG. 18. The in vitro ffLuc activity of drug-resistant Daudi cells was approximately 2700-fold more than untransfected Daudi cells. See FIG. 18.

NOD/scid mice received intraperitoneal adoptive transfer on day 0 of γ-irradiated (FIG. 19, solid line) and non-irradiated (FIG. 19, dashed line) T cells genetically modified with the plasmid ffLucZeo-pcDNA. rHIL-2 (25,000 U/mouse) was given by intraperitoneal injection on day 0. Serial non-invasive biophotonic measurements of the abdomen of these rats are presented as photon flux for a ROI drawn over the abdomen in FIG. 19.

Biophotonic tumor imaging was accomplished as follows. The ffLuc activity from Daudi and human T cells was imaged using a Xenogen IVIS 100 series approximately 15 minutes in anaesthetized mice, placed in the ventral position, after intraperitoneal injection of 150 μL (4.29 mg/mouse) of a freshly thawed aqueous solution of D-luciferin potassium salt (Xenogen, Alameda, Calif.). Each animal was serially imaged at the same time point after D-luciferin administration. Photons emitted from ffLuc⁺ Daudi and T cells for a region of interest (ROI) were quantitated using the software program “Living Image” (Xenogen) and the bioluminescence signal was measured as total photon flux, normalized for exposure time and surface area and expressed in units of photons/second/cm²/steradian. Previous experiments had established that the photon flux from the abdomen was constant within 6.32±8.11%. For anatomical localization, a pseudocolor image representing light intensity (blue, least intense; red, most intense) was superimposed over a digital grayscale body surface reference image.

Statistical methods for analyzing the biophotonic data were as follows. In determining the differences between mouse treatment groups, the primary endpoint used here took into account imaged tumor size across time. By calculating a cumulative area-under-the-curve (AUC) for each mouse, the endpoint generated rewarded the treatments that not only shrank tumors but also kept the tumor small over the course of the study. The mean AUCs between treatments were compared using an exact permutation test using the Hothorn and Hornik R language algorithm in the exactRankTests software package. Details for deriving the permutation p-value in general are discussed in Streitberg and Röhmel. Having obtained the mouse data time points and the photon flux, the connected points were plotted with time on the X-axis and the endpoint on the Y-axis. For any sequential time points, (xᵢ, xⱼ), and their corresponding endpoints, (yᵢ, yⱼ), the area under the curve was calculated using the area of a trapezoid: 0.5*(xⱼ−xⱼ)*(yᵢ+yⱼ). The cumulative AUC for the duration of the experiment was the sum of trapezoids. Cumulative AUCs as an outcome were used to compare results among groups. Using this method, groups with small y-values (i.e., imaged tumor sizes) have small mean AUCs. When a mouse was sacrificed for excessive tumor burden, the last measured tumor size was carried through to the end of the study. As supportive evidence, survival analysis also was performed for this experiment using a threshold of 3.4×10⁶ p/sec/cm²/sr (the mean of the max of mice with no evidence of tumor post day 31 and the min of mice with tumor post day 31) as the threshold for detectable tumor. The time from initial treatment until the bioluminescence fell below the lower threshold defined the “time to remission” endpoint (as used in human trials). Similarly, the durability of remission endpoint was defined as the time from initial remission until tumor growth increased the bioluminescence past the threshold of detection. Based on these endpoints, time until remission and time until tumor recurrence (for mice that had undetectable tumor) was estimated.

Means of cumulative AUCs were compared for each group using the methods described above. The half-life and 90% decay were calculated for each group by estimating each group's total flux mean and interpolating the time in hours when the 50% and 90% threshold was achieved, respectively.

MP1-tetramer⁺Fc⁺ T cells can be stimulated in vivo with AP T-cells to treat established B-lineage tumor. In vitro data demonstrated that MP1-specific T cells are rendered specific for CD19 by the methods described here and that sequential or simultaneous co-exposure of MP1 and CD19 antigens caused a heightened activation state of the bi-specific T cells. Therefore, whether the MP1+AP-T cells could be used to improve control of CD19⁺ tumor in vivo was assessed in a well-recognized murine model.

For the xenograft tumor model, 6- to 10-week-old female NOD/scid (NOD/LtSz-Prkdc^{scid}/J)-mice (Jackson Laboratory, Bar Harbor, Me.) were injected in the peritoneum at day 0 with 5×10⁶ ffLuc⁺ Daudi cells. Beginning on day 7, some of the mice that had engrafted tumor (defined as increasing flux signal) received rhIL-2 (25,000 U/mouse), 20×10⁶ effector T cells, and some of these also received 5×10⁶ γ-irradiated (3,500 cGy) AP-T cells by intraperitoneal (i.p.) injections through 28-gauge hyperdermic needles. (No mice received AP-T cells without effector T cells).

To non-invasively evaluate the anti-tumor activity of the bi-specific T cells in vivo using real-time optical imaging, CD19⁺ Daudi target cells were genetically modified to express ffLuc gene. Serial non-invasive in vivo real-time biophotonic imaging of ffLuc⁺ T cells injected in the peritoneum revealed that by approximately 48 hours, about 90% of the detectable in vivo luciferase activity had diminished from irradiated T cells. The kinetics of loss of luciferase activity was similar for non-irradiated T cells in the absence of antigen, suggesting that the irradiation per se was not the cause for relative loss of luciferase activity. See FIGS. 19 and 20, which show primary human T cells that have been non-invasively imaged in mice by biophotonic detection. Pseudocolor images representing light intensity from γ-irradiated ffLuc⁺T cells in the peritoneum of NOD/scid mice imaged in ventral position are shown in FIG. 20. The luminescence had decreased by 50% by 10 hours and 90% by 48 hours, compared with optical data collected 2 hours after T-cell transfer.

### Example 10

### Biophotonic Imaging of ffLuc⁺ Daudi Before and After Adoptive T-Cell Therapy

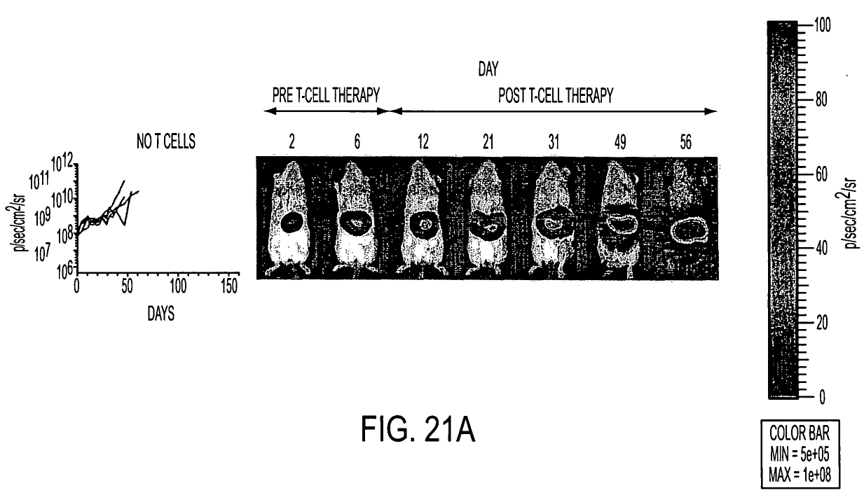

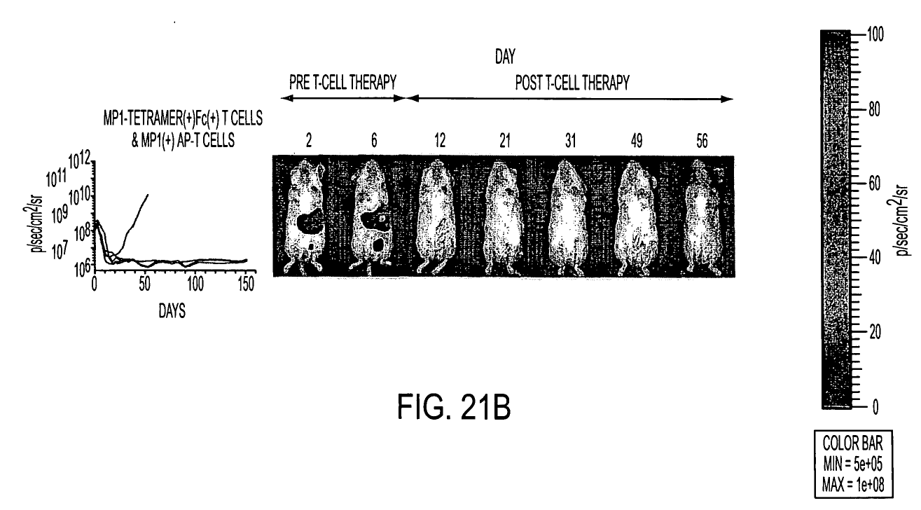

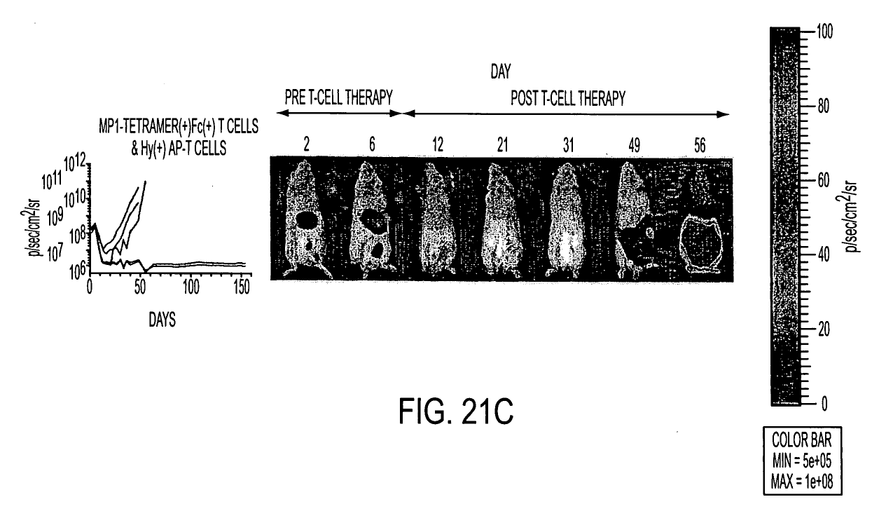

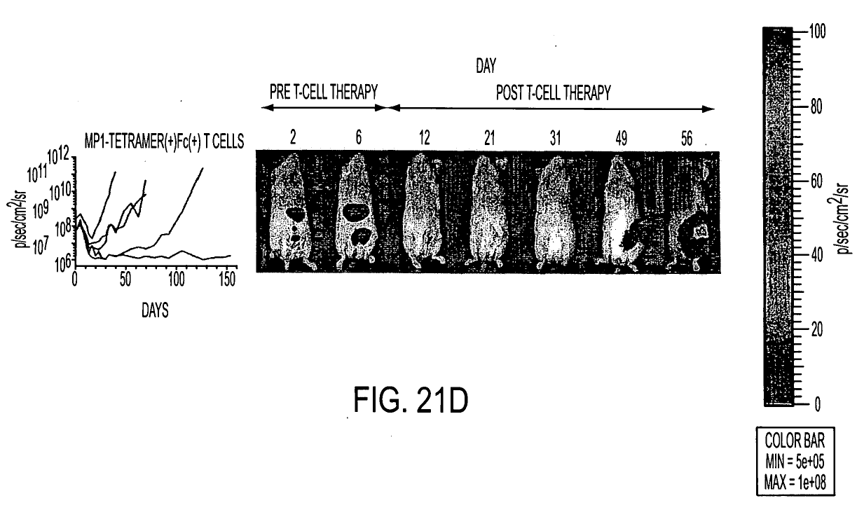

The data in FIG. 21 pertain to NOD/scid mice that were injected intraperitoneally with ffLuc⁺ Daudi cells. Mice that had engrafted with tumor cells (engraftment was defined as two successive biophotonic measurements with increasing ffLuc activity) underwent adoptive immunotherapy using rhIL-2 and MP1-tetramer⁺Fc⁺ T cells alone, or in combination with autologous MP1⁺ AP-T cells or Hy⁺AP-T cells, the latter acting as a antigen^{neg} control. Non-invasive biophotonic imaging measurements revealed the kinetics of tumor growth before and after adoptive immunotherapy. See FIG. 21.

Scatter graphs of tumor flux versus time and pseudocolor images of selected mice (red lines) representing light intensity from ffLuc⁺ Daudi cells in the peritoneum of NOD/scid mice serially imaged in ventral position. On day 0, NOD/scid mice were given 5×10⁶ ffLuc⁺ Daudi cells by intraperitoneal injection. The mice with progressive disease, documented by two concurrent measurements demonstrating increase in tumor flux (measured on days 2 and 6), were divided between 4 treatment groups.

The five mice from group A (FIG. 21A) received no further cellular therapy. On day 7, the five mice in each of groups B (FIG. 21B), C (FIG. 21C), and D (FIG. 21D) received 20×10⁶ MP1-tetramer⁺Fc⁺CD8⁺ T cells by intraperitoneal injection. Mice from group D received additional injections of 20×10⁶ MP1-tetramer⁺Fc⁺CD8⁺ T cells on days 9 and 12. On days 7, 9, 12, 21, 23, and 25 the mice in groups B and C received separate intraperitoneal injections of 5×10⁶ γ-irradiated, thawed autologous hygromycin-resistant AP-T cells that had been genetically modified with HyMP1-pMG (FIG. 21B) or pMG\hat{ }pac (FIG. 21C) coding for HyMP1 and Hy, respectively.

All mice received rhIL-2 (25,000 U/mouse) by separate intraperitoneal injection on days 7, 9, 12, 21, 23, 25. Each mouse was imaged at the same relative time point after D-luciferin administration, which was within 19 minutes after injection. Data are presented as photon flux for a ROI drawn over the whole mouse.

### Example 11

### In Vivo Treatment of Mice

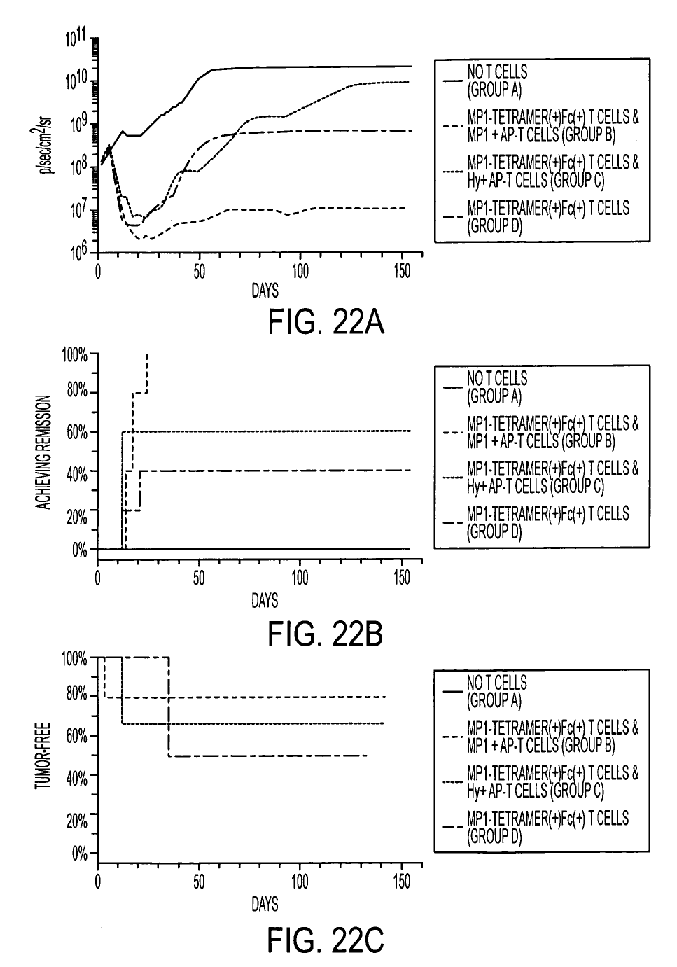

Treatment groups for FIGS. 22A, 22B and 22C are as described for Example 10. Background flux measurements, simultaneously measured from mice without ffLuc⁺ tumor but receiving D-luciferin was 10⁶ to 10⁷ photons/second/cm²/sr. Tumor flux was measured periodically using the methods discussed above. See FIG. 22A. Low tumor flux corresponds to low tumor volume. The group trendlines were derived by smoothing the tumor flux over each mouse within a given group.

Data from mice that achieved complete remission are shown in FIG. 22B. Complete remission was defined as a measurable flux lower than the minimum threshold of tumor detection. This threshold is approximately 3.4×10⁶ p/sec/cm²/sr using the methods described. Time to remission was calculated from the beginning of the experiment until the first date when tumor measurement fell below the detection threshold. Data from progression-free or tumor-free mice are shown in FIG. 22C. Progression-free mice were defined as mice who 1) achieved complete remission, and 2) maintained undetectable tumor measurements until the tumor flux exceeded the threshold from new tumor growth.

The p-value was 0.0503 comparing group B with combined groups C and D. From this, therefore, the mice in group B had more tumor shrinkage and a longer duration of remission than the combined groups C and D. Compared with mice receiving no adoptive immunotherapy, but receiving rhIL-2, there was significant (p=0.051) control of tumor growth. See FIG. 22A. This translated into improved time to progression as well. See FIG. 22B. Mice that did not receive HyMP1⁺ AP-T cells had a relative lack of disease-free survival (p<0.06) compared to mice that received MP1⁺ AP-T cells. See FIG. 22C. These data confirm that the MP1⁺ AP-T cells not only are able to stimulate MP1-specific T cells ex vivo, but improve the effector function of MP1-tetramer⁺Fc⁺ effector T cells in vivo to achieve a greater anti-tumor effect than can be achieved using the effector cells alone.

### Example 12

### CMV-Specific T Cells

T cells expressing the HyCMVpp65 fusion gene are prepared using 16×10⁶ of PBMC, re-suspended at 20×10⁶ cells/ml in hypo-osmolar solution in two cuvettes that are electroporated in the presence of 10 μg of linearized plasmid per cuvette. Following a 10-minute incubation at room temperature the cells are washed and co-cultured in T-75 flasks with T-cell growth media (RPMI 1640 supplemented with 25 mM HEPES and 10% FCS) containing 30 ng/ml OKT3, 50×10⁶ irradiated PBMC and 10×10⁶ irradiated LCL. IL-2 at 25 U/ml is added every 48 hours beginning 24 hours after electroporation. Cytocidal concentrations of hygromycin B at 0.2 mg/ml are added on the fifth day of culture. Every 14 days of culture the genetically modified T cells are expanded in the presence of cytocidal concentrations of neomycin by stimulating with OKT3, irradiated PBMC, irradiated LCL and IL-2. The CMV pp65 protein can be identified by Western Blot analysis of hygromycin T cells, which can be readily expanded and then used to selectively stimulate CMV pp65-specific T cells.

### Example 13

### T Cells Bi-Specific for CD19 and A Virus

T cells bi-specific for CD19R and either MP1 or CMV are prepared as described above. These cells can then be rendered bi-specific using non-viral gene transfer techniques to express the CD19-specific chimeric immunoreceptor (CD19R).

The non-viral gene transfer of the DNA plasmid, co-expressing the CD19R and HyTK selection/suicide genes, into viral-specific T cells can be accomplished using 16×10⁶ of T cells, re-suspended at 20×10⁶ cells/ml in hypo-osmolar solution in two cuvettes that are electroporated in the presence of 10 μg of linearized plasmid per cuvette. Following a 10-minute incubation at room temperature the cells are washed and co-cultured in T-75 flasks with T-cell growth media (RPMI 1640 supplemented with 25 mM HEPES and 10% FCS) containing 30 ng/ml OKT3, 50×10⁶ irradiated PBMC and 10×10⁶ irradiated LCL. IL-2 at 25 U/ml is added every 48 hours beginning 24 hours after electroporation. Cytocidal concentrations of hygromycin B at 0.2 mg/ml is added on the 5^{th} day of culture. Every 14 days of culture the genetically modified T cells are expanded in the presence of cytocidal concentrations of neomycin by stimulating with OKT3, irradiated PBMC, irradiated LCL and IL-2.

### Example 14

### Clinical Study of Bi-Specific T Cells

A phase I study is opened to enroll research participants undergoing a allogeneic HSCT for ALL in CR≧³2 to establish the safety of adoptive therapy with donor-derived bi-specific T cell clones that are (a) CMV- and CD19-bi-specific and (b) EBV- and CD19-bi-specific, and (c) MP1- and CD19-bi-specific. These patients have a rate of relapse of >50% (Appelbaum, 1997; Zwaan et al., 1984; Schmitz et al., 1988) and are at high risk for opportunistic infections with CMV and EBV. PBMC from the donor are stimulated with autologous T cells presenting the desired viral antigen to enrich for viral-specific T cells. The bulk T cell population is genetically modified by electroporation with the plasmid DNA construct encoding for the CD19R and HyTK. Bi-specific T cells are cloned by limiting-dilution. Following ex vivo expansion of T cell clones that recognize both viral antigens and CD19⁺ targets, a series of four escalating cell doses of bi-specific T cells are infused weekly into the recipient, beginning at 1×10⁹ cells/m² and cumulating at 4×10⁹ cells/m². Exogenous low-dose (5×10⁵ IU/m²/dose q 12-hrs) subcutaneous recombinant human interleukin 2 (rhIL-2) may be utilized to support the in vivo persistence of transferred CD8⁺ clones following the 2^{nd}, 3^{rd}, and 4^{th} T cell infusions. Infusions of donor-derived viral-presenting T cells will be used to maintain the in vivo survival of the bi-specific T cells. It is recognized that donor-derived T cells specific for CD19 also target normal CD19⁺ cells of the B cell lineage, but after immunotherapy it is expected that patients will either recover B cell function or humoral immune immunity will be maintained using intravenous immunoglobulin.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

Publications and other materials may illuminate the background of the invention or provide additional details respecting the practice of the invention. The following references are hereby incorporated by reference in their entirety, and for convenience are grouped in the bibliography below.

### BIBLIOGRAPHY

1. Yee, C. et al., “Adoptive T cell therapy using antigen-specific CD8+ T cell clones for the treatment of patients with metastatic melanoma: in vivo persistence, migration, and antitumor effect of transferred T cells,” Proc. Natl. Acad. Sci. U.S.A. 99:16168-16173, 2002.
2. Walter, E. A. et al., “Reconstitution of cellular immunity against cytomegalovirus in recipients of allogeneic bone marrow by transfer of T-cell clones from the donor,” N. Engl. J. Med. 333:1038-1044, 1995.
3. Dudley, M. E. et al. “Cancer regression and autoimmunity in patients after clonal repopulation with antitumor lymphocytes,” Science 298:850-854, 2002.
4. Brodie, S. J. et al., “HIV-specific cytotoxic T lymphocytes traffic to lymph nodes and localize at sites of HIV replication and cell death,” J. Clin. Invest. 105:1407-417, 2000.
5. Heslop, H. E. “Long-term restoration of immunity against Epstein-Barr virus infection by adoptive transfer of gene-modified virus-specific T lymphocytes,” Nat. Med. 2:551-555, 1996.
6. Wang, R. F. et al., “Human tumor antigens for cancer vaccine development,” Immunol. Rev. 170:85-100, 1999.
7. Pardoll, D. “Does the immune system see tumors as foreign or self?” Annu. Rev. Immunol. 21:807-839, 2003.
8. Garrido, F. et al., “MHC antigens and tumor escape from immune surveillance,” Adv. Cancer Res. 83:117-158, 2001.
9. Pule, M. et al., “Artificial T-cell receptors,” Cytotherapy 5:211-226, 2003.
10. Gross, G. et al., “Endowing T cells with antibody specificity using chimeric T cell receptors,” FASEB J. 6:3370-3378, 1992.
11. Jensen, M. C. et al., “Engineered CD20-specific primary human cytotoxic T lymphocytes for targeting B-cell malignancy,” Cytotherapy 5:131-138, 2003.
12. Sadelain, M. et al., “Targeting tumours with genetically enhanced T lymphocytes,” Nat. Rev. Cancer 3:35-45, 2003.
13. Lupton, S. D. et al., “Dominant positive and negative selection using a hygromycin phosphotransferase-thymidine kinase fusion gene,” Mol. Cell. Biol. 11:3374-3378, 1991.
14. Ho, S. N. et al., “Site-directed mutagenesis by overlap extension using the polymerase chain reaction,” Gene 77:51-59, 1989.
15. Mahmoud, M. S. et al., “Enforced CD19 expression leads to growth inhibition and reduced tumorigenicity,” Blood 94:3551-3558, 1999.
16. Gotch, F. et al., “Cytotoxic T lymphocytes recognize a fragment of influenza virus matrix protein in association with HLA-A2,” Nature 326:881-882, 1987.
17. Larsson, M. et al., “Requirement of mature dendritic cells for efficient activation of influenza A-specific memory CD8+ T cells,” J. Immunol. 165:1182-1190, 2000.
18. Morrison, J. et al., “Identification of the nonamer peptide from influenza A matrix protein and the role of pockets of HLA-A2 in its recognition by cytotoxic T lymphocytes,” Eur. J. Immunol. 22:903-907, 1992.
19. David, H. A. “Robust estimation in the presence of outliers,” Robustness in Statistics 1:61-74, 1979.
20. Guttman, I. et al., “Investigation of rules for dealing with outliers in small samples from the normal distribution I: estimation of the mean,” Technometrics 11:527-550, 1969.
21. R Development Core Team. “R: A language and environment for statistical computing,” Vienna, Austria: R Foundation for Statistical Computing; 2003.
22. Hothorn, T. “exactRankTests package for R,” Available at http://cran.r-project.org, 2003.
23. Streitberg, B. et al., “Exact distributions for permutations and rank tests: an introduction to some recently published algorithms,” Statistical Software Newsletter 12:10-17, 1986.
24. Shih, I. M. et al., “Assessing tumors in living animals through measurement of urinary beta-human chorionic gonadotropin,” Nat. Med. 6:711-714, 2000.
25. Gotch, F. et al., “Recognition of influenza A matrix protein by HLA-A2-restricted cytotoxic T lymphocytes. Use of analogues to orientate the matrix peptide in the HLA-A2 binding site,” J. Exp. Med. 168:2045-2057, 1988.
26. Latron, F. et al., “Positioning of a peptide in the cleft of HLA-A2 by complementing amino acid changes,” Proc. Natl. Acad. Sci. U.S.A. 88:11325-11329, 1991.
27. Stewart-Jones, G. B. et al., “A structural basis for immunodominant human T cell receptor recognition,” Nat. Immunol. 4:657-63, 2003.
28. Schultze, J. et al., “B7-mediated costimulation and the immune response,” Blood Rev. 10:111-127, 1996.
29. Young, J. W. et al., “The hematopoietic development of dendritic cells: a distinct pathway for myeloid differentiation,” Stem Cells 14:376-387, 1996.
30. Lehner, P. J. et al., “Human HLA-A0201-restricted cytotoxic T lymphocyte recognition of influenza A is dominated by T cells bearing the V beta 17 gene segment,” J. Exp. Med. 181:79-91, 1995.
31. Lawson, T. M. et al., “Influenza A antigen exposure selects dominant Vbeta17+ TCR in human CD8+ cytotoxic T cell responses,” Int. Immunol. 13:1373-1381, 2001.
32. Moss, P. A. et al., “Extensive conservation of alpha and beta chains of the human T-cell antigen receptor recognizing HLA-A2 and influenza A matrix peptide,” Proc. Natl. Acad. Sci. U.S.A. 88:8987-8990, 1991.
33. Krummel, M. F. et al., “Dynamics of the immunological synapse: finding, establishing and solidifying a connection,” Curr. Opin. Immunol. 14:66-74, 2002.
34. Rojo, J. M. et al., “Physical association of CD4 and the T-cell receptor can be induced by anti-T-cell receptor antibodies,” Proc. Natl. Acad. Sci. U.S.A. 86:3311-3315, 1989.
35. Berger, C. et al., “Nonmyeloablative immunosuppressive regimen prolongs In vivo persistence of gene-modified autologous T cells in a nonhuman primate model,” J. Virol. 75:799-808, 2001.
36. Riddell, S. R. et al., “T-cell mediated rejection of gene-modified HIV-specific cytotoxic T lymphocytes in HIV-infected patients,” Nat. Med. 2:216-223, 1996.
37. Kershaw, M. H. et al., “Dual-specific T cells combine proliferation and antitumor activity,” Nat. Biotechnol. 20:1221-1227, 2002.
38. Rossig, C. et al., “Epstein-Barr virus-specific human T lymphocytes expressing antitumor chimeric T-cell receptors: potential for improved immunotherapy,” Blood 99:2009-2016, 2002.
39. Roessig, C. et al., “Targeting CD19 with genetically modified EBV-specific human T lymphocytes,” Ann. Hematol. 81(Suppl 2):S42-3, 2002.
40. Ahn, J. H. et al., “Identification of the genes differentially expressed in human dendritic cell subsets by cDNA subtraction and microarray analysis,” Blood 100:1742-1754, 2002.
41. Uetsuki, T. et al., “Isolation and characterization of the human chromosomal gene for polypeptide chain elongation factor-1 alpha,” J. Biol. Chem. 264:5791-5798, 1989.
42. Mahmoud, M. S. et al., “Enforced CD19 expression leads to growth inhibition and reduced tumorigenicity,” Blood 94:3551-3558, 1999.

1. A bi-specific T cell which expresses and bears on its surface a viral antigen T cell receptor and cancer antigen-specific chimeric receptor.

2. The bi-specific T cell of claim 1, wherein the chimeric T cell receptor comprises an intracellular signaling domain, a transmembrane domain and a cancer antigen-specific extracellular domain.

3. The bi-specific T cell of claim 2, wherein the cancer antigen is selected from the group consisting of CD19, CD20, neuroblastoma antigen and IL13.

4. The bi-specific T cell of claim 1, wherein the viral antigen is selected from the group consisting of influenza, EBV, CMV and adenovirus.

5. The bi-specific T cell of claim 2, wherein the viral antigen is selected from the group consisting of influenza, EBV, CMV and adenovirus.

6. The bi-specific T cell of claim 3, wherein the viral antigen is selected from the group consisting of influenza, EBV, CMV, adenovirus.

7. A method for treating cancer in a mammal comprising administering a therapeutically acceptable amount of the bi-specific T cell of any one of claims 1-6.

8. A method of abrogating an untoward B cell function in a mammal comprising administering a therapeutically acceptable amount of the bi-specific T cell of any one of claims 1-6, wherein said cancer antigen-specific chimeric T cell receptor is specific for CD19 or CD20, neuroblastoma antigen or IL-13.

9. The method of claim 8, wherein the untoward B cell function is a B-cell mediated autoimmune disease.

10. The method of claim 8, wherein the B-cell mediated autoimmune disease is lupus or rheumatoid arthritis.

11. The method of claim 7, which further comprises effecting persistence in vivo of the bi-specific T cell by administering to the mammal a stimulatory amount of a viral antigen or T-cells expressing a viral antigen, wherein the viral antigen-specific receptor of the bi-specific T cell is the same as the administered viral antigen.

12. A method for effecting persistence in vivo of the bi-specific T cell of any one of claims 1-6 comprising administering to a mammal a stimulatory amount of a viral antigen or T-cells expressing a viral antigen, wherein the viral antigen-specific receptor of the bi-specific T cell is the same as the administered viral antigen.

13. A method for effecting persistence in vivo of the bi-specific T cell of any one of claims 1-6 comprising administering ganciclovir when the bi-specific T cell co-expresses the HyTK fusion gene.

14. A method for vaccinating patients with a desired antigen by administering T cells genetically modified to express the desired antigen.

15. A method of eliminating bi-specific T cells in vivo by withdrawing administration of the viral antigen recognized by the bi-specific T cell or with-holding viral antigen recognized by the bi-specific T cell.
